(19) United States
(12) Patent Application Publication
(10) Pub. No.: US 2015/0362508 A1
(43) Pub. Date: Dec. 17, 2015

(54) EFFECTOR T CELL RSISTANCE

(71) Applicant: BENAROYA RESEARCH INSTITUTE AT VIRGINIA MASON, Seattle, WA (US)

(72) Inventor: Jane Hoyt Buckner, Seattle, WA (US)

(73) Assignee: Benaroya Research Institute at Virginia Mason, Seattle, WA (US)

(21) Appl. No.: 14/764,899

(22) PCT Filed: Jan. 30, 2014
(86) PCT No.: PCT/US2014/013838

§ 371 Date: Jul. 30, 2015

Related U.S. Application Data
(60) Provisional application No. 61/794,689, filed on Mar. 15, 2013.
(60) Provisional application No. 61/758,721, filed on Jan. 30, 2013.

(51) Int. Cl. G01N 33/68

(52) U.S. Cl. CPC G01N 33/6869; G01N 2333/5412; G01N 2800/52; G01N 2333/47; G01N 2333/7155

(57) ABSTRACT

A method for identifying an autoimmune or demyelinating disease in a subject is described. The method includes (a) FIRST COHORT obtaining a sample comprising CD4^{′} T cells from a subject; (b) determining a level of IL-6 responsiveness of CD4⁺ T cells from the sample; (c) comparing the level of IL-6 responsiveness determined in step (b) with a level of IL-6 responsiveness of CD4⁺ T cells in a sample from a healthy subject; and (d) identifying the subject as having an autoimmune or demyelinating disease based upon an elevated level of IL-6 responsiveness in the subject. Biomarkers include, for example, IL-6Rα and/or pSTAT3. Methods for identifying and characterizing multiple sclerosis and relapsing-remitting multiple sclerosis are also described.

### CROSS-REFERENCES TO RELATED APPLICATIONS

This application claims the benefit of U.S. Patent Application No. 61/794,689, filed Mar. 15, 2013, and U.S. Patent Application No. 61/758,721, filed Jan. 30, 2013, both of which are incorporated herein by reference.

### FIELD OF THE DISCLOSURE

The present disclosure relates to methods for identifying autoimmune or demyelinating disease activity in a subject. The disclosure also relates to methods for predicting and/or monitoring autoimmune or demyelinating disease progression in a subject.

### BACKGROUND

Multiple sclerosis (MS) is a chronic autoimmune disease characterized by inflammatory lesions and axonal loss resulting in demyelination and neurodegeneration in the central nervous system. Neuroinflammation characterized, in part, by CD4⁺ T cell infiltrates is the predominant feature of relapsing-remitting multiple sclerosis (RRMS). This suggests that suppression of pathogenic CD4⁺ T cells by regulatory T cells (Treg) has failed in RRMS. Studies of the Treg in individuals with RRMS have demonstrated differences in Treg number and function (Haas, J., et al., Eur. J. Immunol. 35, 3343-3352 (2005); Venken, K., et al., Immunology 123(1), 79-89 (2008); Viglietta, V., et al., J. Exp. Med. 199, 971-979 (2004)). However, in settings where therapy has increased Treg number, there has been no correlation with therapeutic response, suggesting that a lack of Treg cannot fully explain failed tolerance in these subjects (Oh, U., et al., Arch. Neurol. 66, 471-479 (2009)). Recently, it has become clear that failed tolerance in autoimmunity can be mediated through resistance of effector CD4⁺ T cells (Teff) to suppression by Treg. This has been shown in multiple murine models of autoimmunity including the MRL/1 pr model of lupus, the NOD and DO11.10 RIP-mOVA models of diabetes as well as the EAE model of multiple sclerosis (Korn, T., et al., Nat. Med. 13, 423-431 (2007)). Similar findings have been described in multiple human autoimmune diseases including systemic lupus erythematosus (SLE) (Venigalla, R. K., et al., Arthritis Rheum. 58, 2120-2130 (2008)), type 1 diabetes mellitus (T1D) (Lawson, J. M., et al., Clin. Exp. Immunol. 154, 353-359 (2008); Schneider, A., et al., J. Immunol. 181, 7350-7355 (2008)), rheumatoid arthritis (RA) (van Amelsfort, J. M., et al., Arthritis Rheum. 56, 732-742 (2007)), juvenile idiopathic arthritis (JIA) (Haufe, S., et al., Arthritis Rheum. 63, 3153-3162 (2011); Wehrens, E. J., et al., Blood 118(13), 3538-3548 (2011)) and psoriasis (Goodman, W. A., et al., J. Immunol. 183, 3170-3176 (2009)). Multiple factors have been shown to enhance Teff resistance, including the maturation state or lineage of CD4⁺ T cells (Yang, J., et al., Proc. Natl. Acad. Sci. USA 104, 19954-19959 (2007)) and exposure to the cytokines TNF-α, IL-4, IL-12, IL-6, IL-7, IL-15 and IL-21 (Korn, T., et al., Nat. Med. 13, 423-431 (2007)). Among these, IL-6 has been linked to Teff resistance in the mouse, healthy individuals and in the autoimmune disease psoriasis (Goodman, W. A., et al., J. Immunol. 183, 3170-3176 (2009); Goodman, W. A., et al., J. Immunol. 186(6), 3336-3345 (2011); Pasare, C., and Medzhitov, R., Science 299, 1033-1036 (2003)).

IL-6 levels rise quickly in the setting of inflammation. The T cell response to IL-6 is mediated via the classical signaling pathway in which IL-6 binds to the IL-6Rα on the cell surface, followed by gp130 recruitment to the IL-6/IL-6R complex which then activates and preferentially phosphorylates signal transducers and activators of transcription 3 (STAT3) and to a lesser degree STAT1 (Naugler, W. E., and Karin, M., Trends Mol. Med. 14, 1010-129 (2008)). Alternatively, IL-6 binds sIL-6Rα in the serum forming a complex that can signal through membrane-bound gp130 inducing the phosphorylation of STAT3 and STAT1. The impact of IL-6 on the immune response has the potential to impede tolerogenic responses in several ways. IL-6 has been shown to inhibit apoptosis in T cells (Takeda, K., et al., J. Immunol. 161, 4652-4660 (1998)), it is required for the differentiation of Th17 cells, and local exposure to IL-6 can result in the development of Teff resistant to suppression (Goodman, W. A., et al., J. Immunol. 186(6), 3336-3345 (2011)).

A potential role for IL-6 in MS is suggested by the finding that IL-6 deficient (Samoilova, E. B., et al., J. Immunol. 161, 6480-6486 (1998)) and STAT3 deficient mice (Liu, X., et al., J. Immunol. 180, 6070-6076 (2008)) are resistant to EAE induction. It is further supported by recent genome wide association studies that have shown an association between variants in the STAT3 gene and MS risk (Jakkula, E., et al., Am. J. Hum. Genet. 86, 285-291 (2010)). Evidence that a dysregulated IL-6 pathway is involved in MS pathogenesis is indicated by an increase of IL-6Rα expression on CD4⁺ T cells from MS patients (Bongioanni, P., et al., Eur. J. Neurol. 7, 291-297 (2000)) and by the finding that patients who undergo a relapse have elevated pSTAT3 and pSTAT1 levels (Frisullo, G., et al., J. Neurosci. Res. 84, 1027-1036 (2006)).

### SUMMARY

In accordance with the foregoing, in one aspect, a method for identifying an autoimmune or demyelinating disease in a subject is provided. The method comprises the steps of (a) obtaining a sample comprising CD4⁺ T cells from a subject; (b) determining a level of IL-6 responsiveness of CD4⁺ T cells from the sample; (c) comparing the level of IL-6 responsiveness determined in step (b) with a level of IL-6 responsiveness of CD4⁺ T cells in a sample from a healthy subject; and (d) identifying the subject as having an autoimmune or demyelinating disease based upon an elevated level of IL-6 responsiveness in the subject. In one embodiment, the level of IL-6 responsiveness is determined by a level of IL-6Rα and/or a level of pSTAT3 expression.

In one embodiment, the subject is identified as having multiple sclerosis or relapsing-remitting multiple sclerosis. In one embodiment, the subject has been diagnosed with or treated for multiple sclerosis.

In one embodiment, a course of treatment for a subject having an elevated level of IL-6 responsiveness is more aggressive than a course of treatment for a subject not having an elevated level of IL-6 responsiveness.

In another aspect, a method for predicting autoimmune or demyelinating disease progression in a subject is provided. The method comprises the steps of (a) obtaining a sample comprising CD4⁺ T cells from a subject; (b) determining a level of IL-6 responsiveness of CD4⁺ T cells from the sample; (c) comparing the level of IL-6 responsiveness determined in step (b) with a level of IL-6 responsiveness of CD4⁺ T cells in a sample from a healthy subject; and (d) predicting progression of a disease or condition in the subject based upon an elevated level of IL-6 responsiveness in the subject. In one embodiment, the level of IL-6 responsiveness is determined by a level of IL-6Rα and/or pSTAT3 expression.

In one embodiment, the disease or condition is multiple sclerosis or relapsing-remitting multiple sclerosis. In one embodiment, the subject has been diagnosed with or treated for multiple sclerosis.

In one embodiment, a course of treatment for a subject having an elevated level of IL-6 responsiveness is more aggressive than a course of treatment for a subject not having an elevated level of IL-6 responsiveness.

In another aspect, a method for monitoring autoimmune or demyelinating disease progression in a subject is provided. The method comprises the steps of: (a) obtaining a first sample comprising CD4⁺ T cells from a subject; (b) determining a level of IL-6 responsiveness of CD4⁺ T cells from the first sample; (c) obtaining a second sample comprising CD4⁺ T cells from a subject; (d) determining a level of IL-6 responsiveness of CD4⁺ T cells from the second sample; (e) comparing the level of IL-6 responsiveness of CD4⁺ T cells from the first sample and the second sample; and (f) identifying disease progression in the subject, wherein a change in the level of IL-6 responsiveness is indicative of disease progression. In one embodiment, the level of IL-6 responsiveness is determined by a level of IL-6Rα and/or pSTAT3 expression.

In one embodiment, an increase in the level of IL-6 responsiveness is indicative of the subject having multiple sclerosis or relapsing-remitting multiple sclerosis. In one embodiment, the subject has been diagnosed with or treated for multiple sclerosis.

In one embodiment, a course of treatment for a subject having an increased level of IL-6 responsiveness is more aggressive than a course of treatment for a subject not having an elevated level of IL-6 responsiveness.

In another aspect, a method for predicting disease activity or responsiveness to immunomodulatory therapies in a subject with an autoimmune or demyelinating disease is provided. The method comprises the steps of (a) obtaining a sample comprising CD4⁺ T cells from a subject; (b) exposing at least a portion of CD4⁺ T cells from the sample to IL-6 to provide stimulated CD4⁺ T cells; (c) quantifying an expression level of a biomarker in the stimulated CD4⁺ T cells; and (d) determining if the subject is in an active phase of the autoimmune or demyelinating disease, wherein an increased expression level of the biomarker is indicative of an active phase. In one embodiment, the biomarker is IL-6Rα and/or pSTAT3. In one embodiment, the autoimmune or demyelinating disease is multiple sclerosis or relapsing-remitting multiple sclerosis.

In another aspect, an in vitro method for characterizing relapsing-remitting multiple sclerosis in a subject is provided. The method comprises the steps of (a) obtaining a sample comprising CD4⁺ T cells from a first subject having relapsing-remitting multiple sclerosis; (b) isolating CD4⁺ T cells from the sample to provide isolated CD4⁺ T cells; (c) co-culturing the isolated CD4⁺ T cells with Treg cells obtained from a second subject not having relapsing-remitting multiple sclerosis; (d) quantifying an amount of CD4⁺ T cell proliferation; and (e) determining if CD4⁺ T cell proliferation is suppressed, wherein suppressed CD4⁺ T cell proliferation is indicative of an active phase of relapsing-remitting multiple sclerosis in the first subject.

In another aspect, a method for characterizing relapsing-remitting multiple sclerosis in a subject is provided. The method comprises the steps of (a) obtaining a sample comprising CD4⁺ T cells from a subject having multiple sclerosis; (b) exposing at least a portion of CD4⁺ T cells from the sample to IL-6 to provide stimulated CD4⁺ T cells; (c) quantifying an amount of pSTAT3 in the stimulated CD4⁺ T cells; and (d) determining if the amount of pSTAT3 is increased, wherein increased pSTAT3 is indicative of an active phase of relapsing-remitting multiple sclerosis in a subject.

In another aspect, the use of a compound effective to reduce phosphorylation of STAT3 in the manufacture of a medicament for use in the treatment of multiple sclerosis in a subject in need thereof is provided.

In another aspect, a composition for treating multiple sclerosis comprising a composition selected from the group consisting of (a) an IL-6 inhibitor; (b) an IL-6Rα antagonist; (c) a sIL-6Rα antagonist; and/or (d) a STAT3 phosphorylation blocker, wherein the composition is administered for a time and in an amount effective to treat multiple sclerosis is provided.

### DESCRIPTION OF THE DRAWINGS

The foregoing aspects and many of the attendant advantages of the disclosed methods and compositions will become more readily appreciated as the same become better understood by reference to the following detailed description, when taken in conjunction with the accompanying drawings, wherein:

FIG. 1A graphically represents the percent inhibition of Teff samples obtained from individual control or RRMS subjects in the first cohort.

FIG. 1B graphically represents the percent inhibition of Teff samples obtained from individual control or RRMS subjects in the second cohort.

FIG. 2A graphically represents the percent pSTAT3 (IL-6) for control and RRMS samples obtained from subjects in the first and second cohorts.

FIG. 2B graphically represents the AMFI (IL-6) for samples obtained from control and RRMS subjects in the first cohort.

FIG. 2C graphically represents the percent inhibition for control and RRMS responder cells at a ratio of 1:4 plotted against MFI pSTAT3 in response to IL-6.

FIG. 2D graphically represents the percent inhibition for samples obtained from control and RRMS subjects in the presence or absence of a STAT3 inhibitor.

FIG. 3A graphically represents the AMFI for samples obtained from control and RRMS subjects in the first cohort following activation with IL-27.

FIG. 3B graphically represents the AMFI for samples obtained from control and RRMS subjects in the first cohort following activation with IL-10.

FIG. 3C graphically represents the level of IL-6Rα expression in CD4⁺ T cells obtained from control and RRMS subjects in the first cohort.

FIG. 3D graphically represents the level of gp130 expression in CD4⁺ T cells obtained from control and RRMS subjects in the first cohort.

FIG. 3E graphically represents the relationship between IL-6Rα expression and MFI pSTAT3 levels for samples obtained from control and RRMS subjects, including two RRMS outliers, in response to IL-6 stimulation.

FIG. 4A graphically represents the percent inhibition of Teff samples obtained from individual control or RRMS subjects in the first cohort.

FIG. 4B graphically represents the percent inhibition of Teff samples obtained from individual control or RRMS subjects in the second cohort.

FIG. 4C graphically represents the MFI pSTAT3 for samples obtained from control and RRMS subjects following stimulation with IL-6.

FIG. 5A is a scatterplot that shows gating strategy for calculation of percent inhibition in suppression assay and cytometric analysis of phosphorylated STAT3 and IL-6Rα surface expression.

FIG. 5B is a histogram that illustrates the percent of maximum of CD4⁺ T cells that were gated on live plotted against the level of phosphorylated STAT3 determined based on unstimulated PBMC (red line).

FIG. 5C is a histogram that illustrates the percent of maximum of CD4⁺ T cells that were gated on live plotted against the level of IL-6Rα expression (blue line) based on an isotype control (red line).

FIG. 6A graphically represents the percent inhibition for samples obtained from control and RRMS subjects in the first cohort plotted against the percent proliferation of Teff.

FIG. 6B graphically represents the percent inhibition for samples obtained from control and RRMS subjects in the second cohort plotted against the percent proliferation of Teff.

FIG. 7A graphically represents the level of IL-6 in culture supernatants in samples obtained from control and RRMS subjects.

FIG. 7B graphically represents the level of IL-17 in culture supernatants in samples obtained from control and RRMS subjects.

FIG. 7C graphically represents the level of IFN-γ in culture supernatants in samples obtained from control and RRMS subjects.

FIG. 7D graphically represents the level of TNF-α in culture supernatants in samples obtained from control and RRMS subjects.

FIG. 7E graphically represents the level of IL-2 in culture supernatants in samples obtained from control and RRMS subjects.

FIG. 7F graphically represents the level of IL-1β in culture supernatants in samples obtained from control and RRMS subjects.

FIG. 7G graphically represents the level of IL-4 in culture supernatants in samples obtained from control and RRMS subjects.

FIG. 8 graphically represents the level of basal MFI pSTAT3 expression determined by gating on total CD4⁺ T cells for samples obtained from control and RRMS subjects in the first and second cohorts.

FIG. 9 graphically represents the percent pSTAT3 of CD4⁺ T cells following stimulation with IL-6 or a combination of IL-6 and sIL-6Rα.

FIG. 10A is a schematic model for a representative intracellular IL-17 staining

FIG. 10B graphically represents the percent IL-17 of CD4⁺ T cells for samples obtained from control and RRMS subjects.

FIG. 10C graphically represents the percent inhibition for samples obtained from control and RRMS subjects characterized as IL-17 high and IL-17 low.

FIG. 11 graphically represents the percent inhibition for samples of CD4⁺CD25⁻ T cells obtained from control subjects and cultured in the absence or presence of IL-6.

FIG. 12 graphically represents the level of sIL-6Rα in the serum of subjects in the first cohort plotted against the level of IL-6Rα surface expression.

### DETAILED DESCRIPTION

Unless specifically defined herein, all terms used herein have the same meaning as they would to one skilled in the art of the present disclosure. The following definitions are provided in order to provide clarity with respect to the terms as they are used in the specification and claims to describe the present disclosure.

As used herein, the term “regulatory T cells” or “Treg” refers to T cells that express the cell surface markers CD4 and CD 25 (e.g., T cells that are CD4⁺ and CD25⁺), and which express FoxP3 protein as measured by a Western blot and/or FoxP3 mRNA transcript (e.g., T cells that are FoxP3⁺).

As used herein, the term “effector T cells” or “Teff” refers to effector T cells that express the cell surface marker CD4 (e.g., T cells that are CD4⁺) and do not express FoxP3 protein (e.g., T cells that are FoxP3⁻).

As used herein, the term “tolerance” includes refractivity to activating receptor-mediated stimulation. Such refractivity is generally antigen-specific and persists after exposure to the tolerizing antigen has ceased. For example, tolerance is characterized by lack of cytokine production, e.g., IL-2. Tolerance can occur to self-antigens or to foreign antigens.

As used herein, the term “peptide” or “polypeptide” is a linked sequence of amino acids and may be natural, recombinant, synthetic or a modification or combination of natural, synthetic, and recombinant.

As used herein the term “treating” refers to preventing, suppressing, repressing or eliminating the disease or demyelinating condition. Preventing the disease or condition involves administering a composition of the present disclosure to a subject prior to onset of the disease. Suppressing the disease or condition involves administering a composition of the present disclosure to a subject after induction of the disease or condition but before its clinical appearance. Repressing a disease or condition involves administering a composition of the present disclosure to a subject after clinical appearance of the disease or condition.

As used herein, the expression “therapeutically effective amount” refers to an amount of the composition which is effective to achieve a desired therapeutic result, such as, for example, the prevention, amelioration or prophylaxis of an autoimmune disease or inflammatory condition.

As used herein, the term “autoimmune disease” refers to a disease or disorder arising from and directed against an individual's own tissues. Examples of autoimmune diseases or disorders include, but are not limited to multiple sclerosis (MS), relapsing-remitting multiple sclerosis (RRMS), arthritis (rheumatoid arthritis (RA), juvenile rheumatoid arthritis, psoriatic arthritis), conditions involving infiltration of T cells and chronic inflammatory responses, autoimmune myocarditis, pemphigus, and type 1 diabetes.

As used herein, the term “stimulation” refers to a primary response induced by ligation of a cell surface moiety. For example, in the context of receptors, such stimulation entails the ligation of a receptor and a subsequent signal transduction event. With respect to stimulation of a T cell, such stimulation refers to the ligation of a T cell surface moiety that in one embodiment subsequently induces a signal transduction event. Further, the stimulation event may activate a cell and upregulate or downregulate expression or secretion of a molecule. Thus, ligation of cell surface moieties, even in the absence of a direct signal transduction event, can result in the reorganization of cytoskeletal structures, or in the coalescing of cell surface moieties, each of which could serve to enhance, modify, or alter subsequent cell responses.

As used herein, the term “activation” refers to the state of a cell following sufficient cell surface moiety ligation to induce a noticeable biochemical or morphological change. Within the context of T cells, such activation refers to the state of a T cell that has been sufficiently stimulated to induce cellular proliferation. Activation of a T cell may also induce cytokine production and performance of regulatory or cytolytic effector functions. Within the context of other cells, this term infers either up or down regulation of a particular physico-chemical process.

As used herein, the term “protein” includes proteins, polypeptides and peptides; and can be an intact molecule, a fragment thereof, or multimers or aggregates of intact molecules and/or fragments; and can occur in nature or be produced, e.g., by synthesis (including chemical and/or enzymatic) or genetic engineering.

As used herein, the terms “agent,” “ligand,” or “agent that binds a cell surface moiety” refer to a molecule that binds to a defined population of cells. The agent may bind any cell surface moiety, such as a receptor, an antigenic determinant, or other binding site present on the target cell population. The agent can be a protein, peptide, antibody and antibody fragments thereof, fusion proteins, synthetic molecule, an organic molecule (e.g., a small molecule), or the like. Within the specification and in the context of T cell stimulation, antibodies are used as a prototypical example of such an agent.

As used herein, “immune response or responsiveness” refers to activation of cells of the immune system, including but not limited to, T cells, such that a particular effector function(s) of a particular cell is induced. Effector functions can include, but are not limited to, proliferation, secretion of cytokines, secretion of antibodies, expression of regulatory and/or adhesion molecules, and the ability to induce cytolysis.

As used herein, “stimulating an immune response” refers to any stimulation such that activation and induction of effector functions of cells of the immune system are achieved.

The term “exposing” as used herein, refers to bringing into the state or condition of immediate proximity or direct contact.

CD4⁺ Teff cells are resistant to regulation via FOXP3⁺ Tregs in RRMS. To determine whether Teff resistance is present in individuals with RRMS, blood samples were obtained from a cohort of RRMS subjects not taking any immunomodulatory therapy within three months of blood draw and compared them to samples from healthy controls. TABLE 1 shows clinical evaluations for the first cohort of untreated RRMS patients and healthy individuals, and TABLE 2 shows clinical evaluations for the second cohort of untreated RRMS patients and healthy individuals.

TABLE 1
Clinical evaluation for first cohort of untreated RRMS patients

| RRMS | | | | | Disease |
|---|---|---|---|---|---|
| patient | Gender* | Age | Flaring^{†} | Comments | Activity^{††} |
| 1 | M | 57 | No | | No |
| 2 | F | 42 | No | | Yes |
| 3 | M | 41 | No | | Yes |
| 4 | F | 68 | No | | No |
| 5 | F | 42 | No | | Yes |
| 6 | F | 32 | No | | Yes |
| 7 | F | 61 | No | enhancing lesions | Yes |
| | | | | MRI 1 week prior | |
| 8 | M | 30 | No | | Yes |
| 9 | F | 27 | No | enhancing lesions | Yes |
| | | | | MRI on 4 days later | |
| 10 | F | 43 | No | | No |
| 11 | F | 49 | No | | Yes |
| 12 | F | 45 | Yes | | Yes |
| 13 | F | 58 | No | | No |
| 14 | F | 45 | No | enhancing lesions | Yes |
| | | | | MRI on 10 days | |
| | | | | later | |

Healthy individuals for first cohort

| Controls | Gender* | Age |
|---|---|---|
| 1 | M | 58 |
| 2 | M | 33 |
| 3 | M | 25 |
| 4 | M | 28 |
| 5 | F | 23 |
| 6 | M | 31 |
| 7 | M | 27 |
| 8 | M | 29 |
| 9 | M | 27 |
| 10 | M | 40 |
| 11 | F | 28 |

*gender: M = male; F = female
^{†}flaring: clinical symptoms
^{††}disease activity: Yes = active RRMS; No = inactive RRMS

TABLE 2
Clinical evaluation for second cohort of untreated RRMS patients

| RRMS | | | | | Disease |
|---|---|---|---|---|---|
| patient | Gender* | Age | Flaring^{†} | Comments | Activity^{††} |
| 1 | F | 22 | Yes | newly diagnosed | Yes |
| 2 | F | 61 | No | | No |
| 3 | F | 26 | No | newly diagnosed | Yes |
| 4 | F | 31 | No | newly diagnosed | Yes |
| 5 | F | 52 | No | | No |
| 6 | F | 51 | No | | No |
| 7 | F | 37 | No | | Yes |
| 8 | F | 24 | No | | No |
| 9 | M | 50 | No | newly diagnosed | Yes |
| 10 | F | 44 | No | | Yes |

Healthy individuals for second cohort

| Controls | Gender* | Age |
|---|---|---|
| 1 | F | 43 |
| 2 | F | 25 |
| 3 | F | 50 |
| 4 | M | 38 |
| 5 | F | 29 |
| 6 | F | 34 |
| 7 | F | 26 |
| 8 | F | 52 |
| 9 | F | 23 |
| 10 | F | 28 |
| 11 | M | 27 |
| 12 | M | 38 |
| 13 | M | 38 |

*gender: M = male; F = female
^{†}flaring: clinical symptoms
^{††}disease activity: Yes = active RRMS; No = inactive RRMS

The level of Teff suppression was measured using an in vitro assay in which the CD4⁺CD25⁻ T cells of patients with RRMS were co-cultured with in vitro generated Treg from healthy subjects at a Treg to Teff ratio of 1:4 in the presence of anti-CD3/anti-CD28 coated beads and where proliferation is measured using CFSE. It has been previously demonstrated that in vitro generated CD4⁺CD25^{bright}FOXP3⁺ T cells, termed adaptive Treg (aTregs), suppress Teff responses in a contact dependent manner and that they have a similar suppressive capacity as nTreg in this assay system (Schneider, A., et al., J. Immunol. 181, 7350-7355 (2008); Long, S. A., and Buckner, J. H., J. Autoimmun. 30, 293-302 (2008); Walker, M. R., et al., Proc. Natl. Acad. Sci. USA 102, 4103-4108 (2005)). Using this approach, it was found that suppression of RRMS Teff cells (n=14; mean 55%, SD=15.0) was significantly lower than that seen for control Teff (n=11; mean 75%, SD=6.6) (p=0.0003).

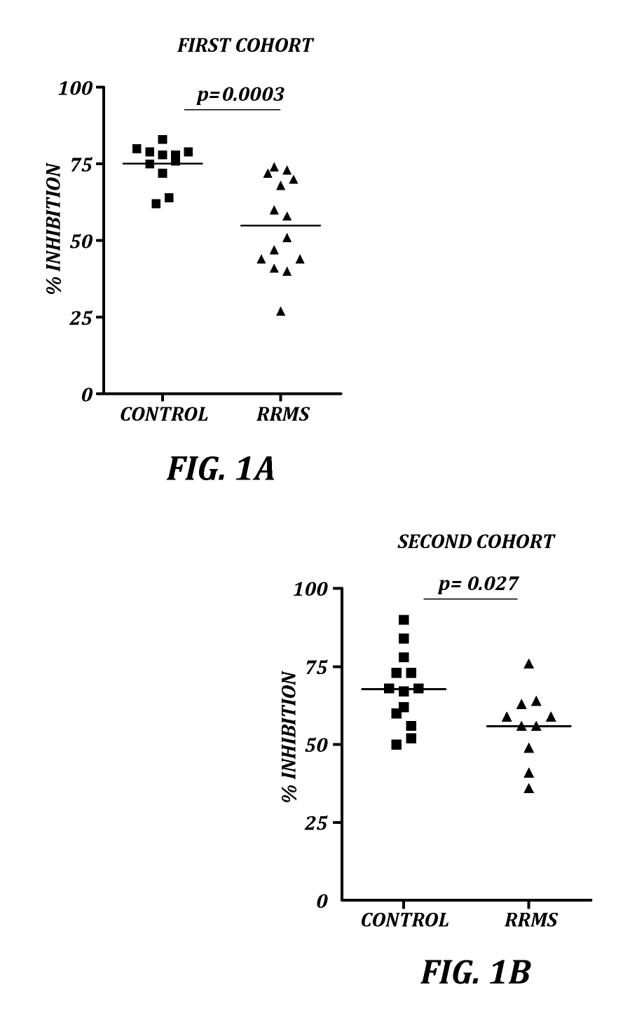

FIGS. 1A and 1B show the Teff of RRMS patients are resistant to Treg-mediated suppression. Control aTreg were co-cultured at a ratio of 1:4 with either Teff from Controls or RRMS individuals. CFSE labeled cells were activated in the presence of anti-CD3/anti-CD28 coated beads for 4 days followed by FACS analysis. Percent inhibition was determined for the first cohort (FIG. 1A) and the second cohort (FIG. 1B). Symbols represent individual control (square) and RRMS (triangle) subjects. Statistical significance was determined using an independent Student t-test with the Welch's correction (Schneider, A., and Buckner, J. H., Methods Mol. Biol. 707, 233-241 (2011)).

To confirm the association of Teff resistance in RRMS patients, a second independent cohort of untreated RRMS subjects was selected for study. As with the first cohort of subjects, RRMS Teff cells (n=10) demonstrated a significant impairment in Treg-mediated suppression when compared with healthy individuals (n=13; p=0.027; see FIG. 1B).

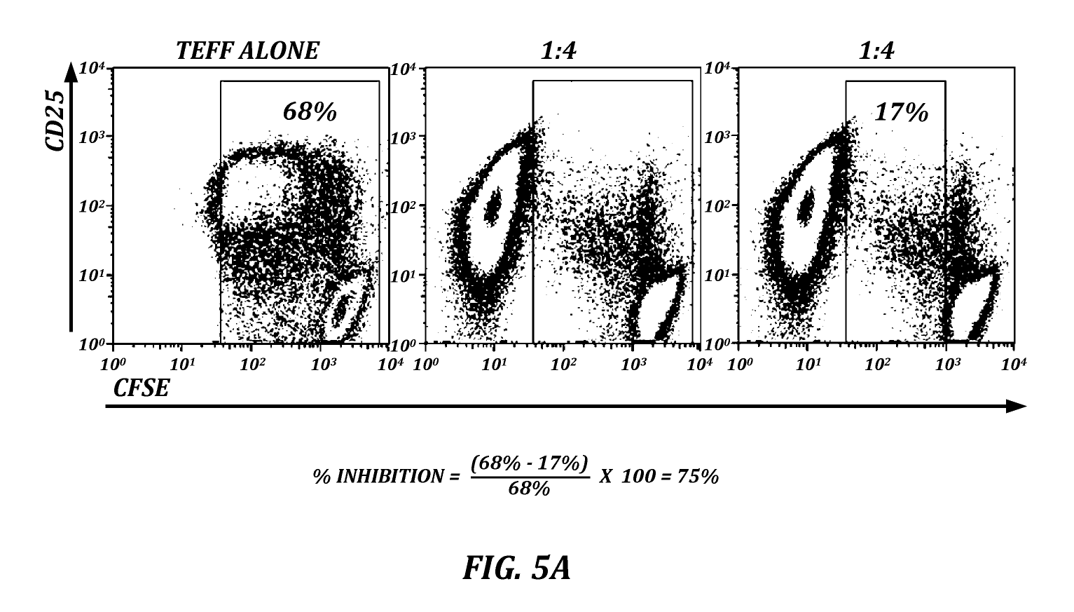

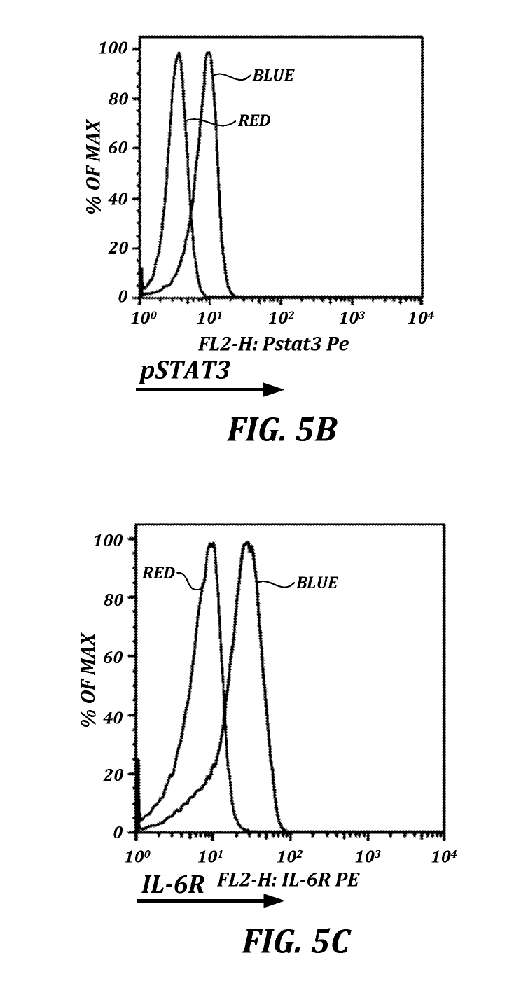

FIGS. 5A, 5B, and 5C relate to a gating strategy for calculation of percent inhibition in a suppression assay and a cytometric analysis of phosphorylated STAT3 and IL-6Rα surface expression. Referring to FIG. 5A, FACS analysis was performed on day 4 by gating on live cells and excluding the CFSE low population (unstained Treg) to determine the percent of the responder cells that have diluted CFSE. The percent inhibition was determined by comparing the percent of proliferating Teff cells cultured alone to the percent of proliferating Teff cells in co-culture with in vitro generated Treg at a ratio 1:4 (Treg:responder cells).

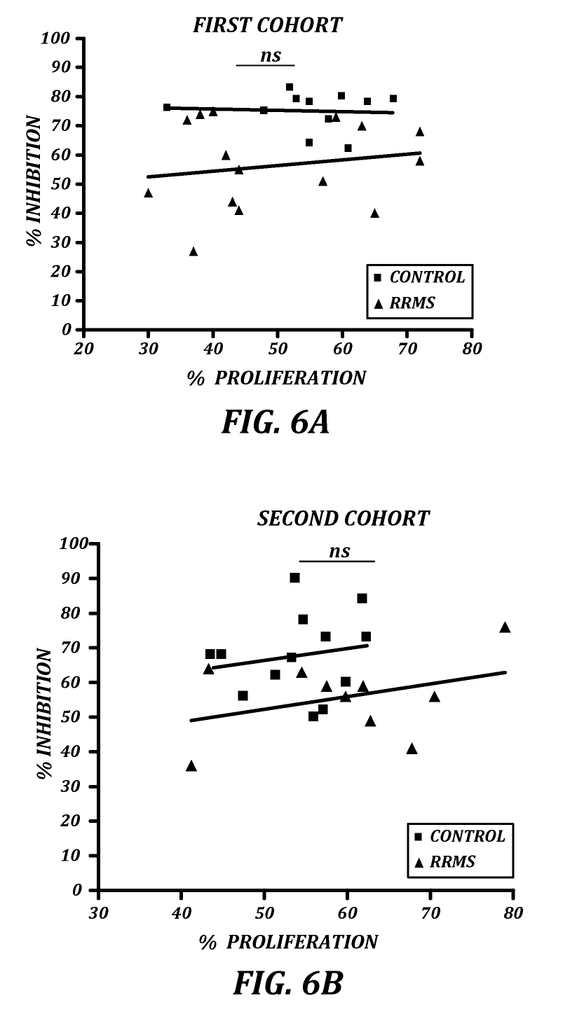

Multiple factors including extrinsic factors such as the strength of stimulation and the cytokine environment as well as intrinsic factors such as the lineage and maturation state of the Teff (Yang, J., et al., Proc. Natl. Acad. Sci. USA 104, 19954-19959 (2007)) contribute to Teff resistance. Variation in the level of suppression in these assays did not appear to be due to differences in the strength of activation based on the finding that proliferation of Teff in the absence of Treg did not differ between RRMS and control subjects (first cohort: mean 55%, SD=9.3, mean 50%, SD=14.1; second cohort: mean 54%, SD=6.0, mean 60%, SD=11.6) and no correlation was present between percent proliferation and inhibition. FIGS. 6A and 6B show percent inhibition does not correlate with baseline proliferation. Referring to FIGS. 6A and 6B, percent inhibition for controls and RRMS is plotted against percent proliferation of Teff when cultured alone and activated with anti-CD3/anti-CD28 coated beads, followed by FACS analysis on day 4 for the first cohort (FIG. 6A) and the second cohort (FIG. 6B). Further, when the composition of the Teff population was evaluated, there was no difference in the percentage of CD4⁺CD45RO⁺ memory cells among the Teff population of RRMS as compared to control subjects (mean 54%, SD=14.0; mean 52%, SD=13.3).

Thus, in one aspect, a method for characterizing relapsing-remitting multiple sclerosis in a subject is provided. The method comprises the steps of (a) obtaining a sample comprising CD4⁺ T cells from a first subject having relapsing-remitting multiple sclerosis; (b) isolating CD4⁺ T cells from the sample to provide isolated CD4⁺ T cells; (c) co-culturing the isolated CD4⁺ T cells with Treg cells obtained from a second subject not having relapsing-remitting multiple sclerosis; (d) quantifying an amount of CD4⁺ T cell proliferation; and (e) determining if CD4⁺ T cell proliferation is suppressed, wherein suppressed CD4⁺ T cell proliferation is indicative of an active phase of relapsing-remitting multiple sclerosis in the first subject.

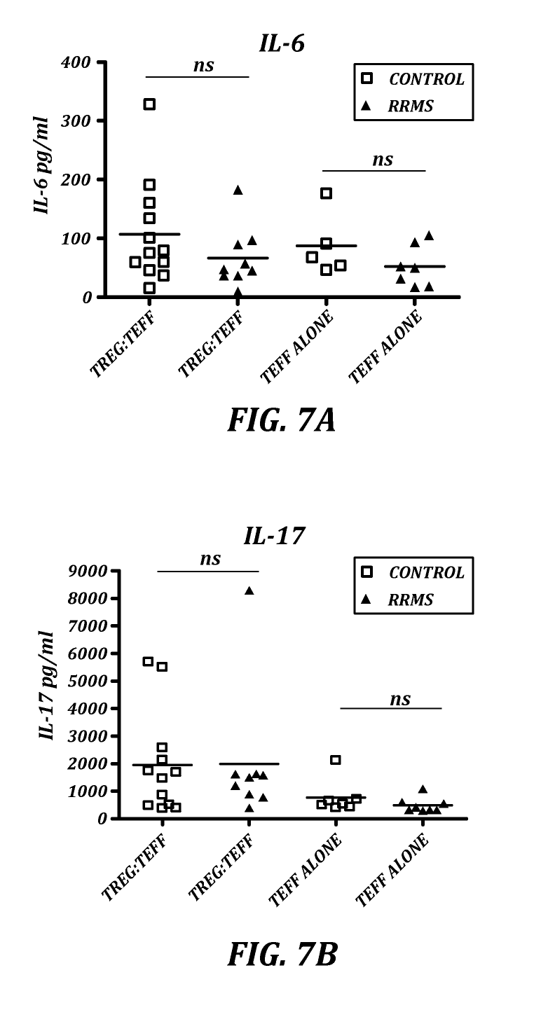

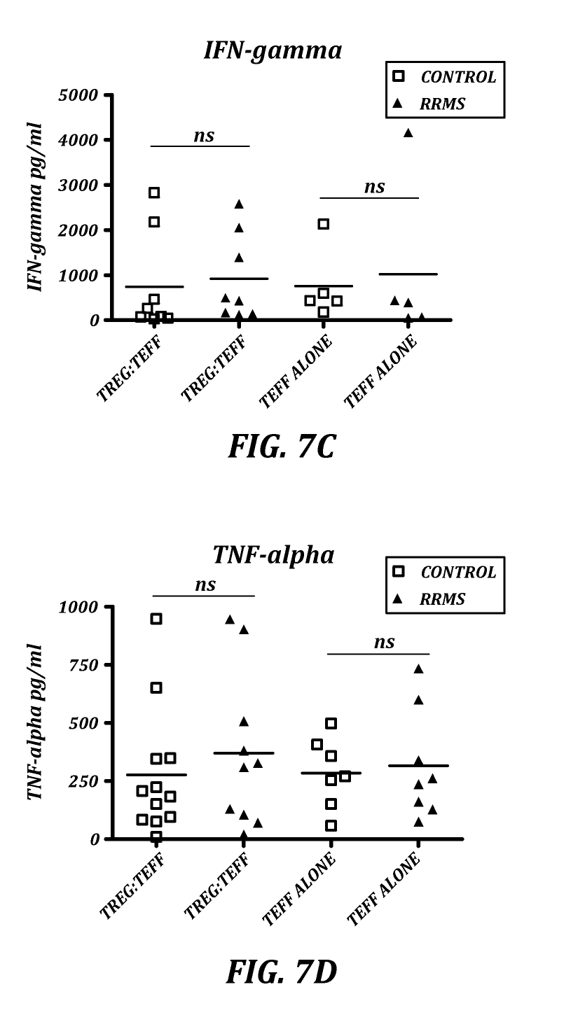

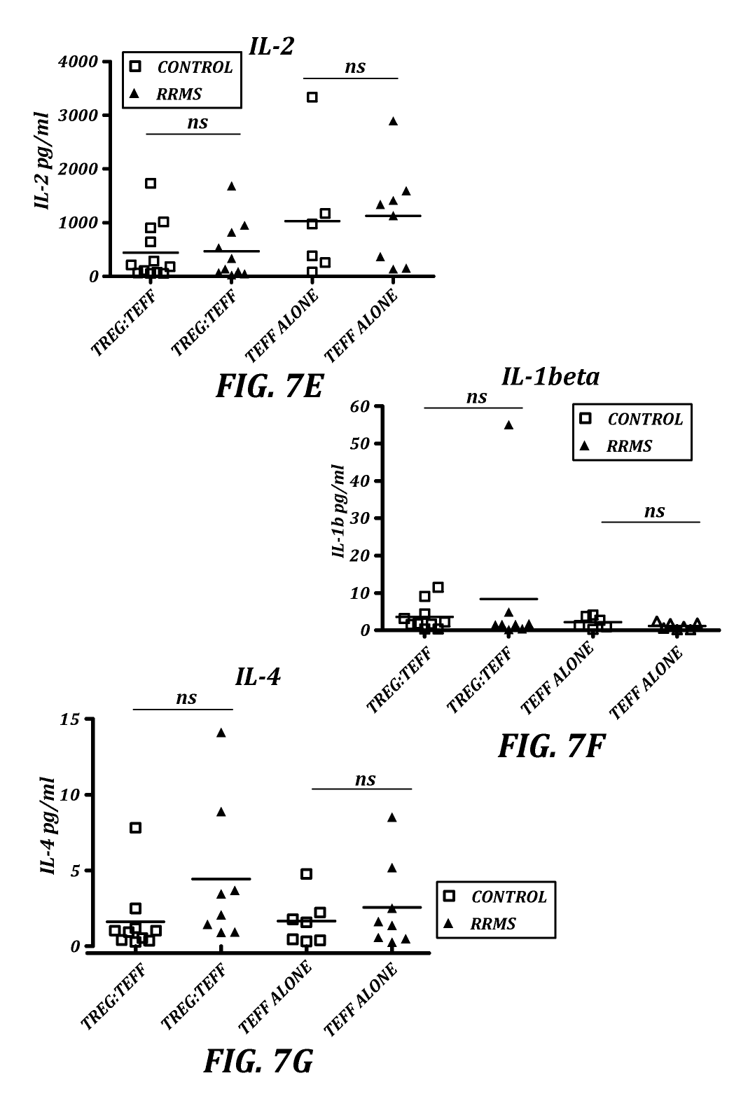

The possibility that the cytokine milieu in the co-culture assays may contribute to the resistance that was found among control and RRMS subjects was examined. Culture supernatants taken at 48 hours from co-cultures and Teff cells alone activated by beads were tested for cytokines using a Luminex based assay. FIGS. 7A-7G show a Luminex analysis of culture supernatants. Referring to FIGS. 7A-7G, cytokines were determined in control and RRMS subject samples taken after 48 hours from the co-culture or when Teff cells were cultured alone and activated with beads. The cytokines IL-1β, IL-2, IL-4, IL-6, IL-17A, IFN-γ and TNF-α were detected in the supernatants, however, there was no significant difference between the cytokine profiles obtained for patient groups or controls.

To address the systemic impact of cytokines, the serum from RRMS subjects was examined, and the following cytokines were detected: IL-1α, IL-1β, TNF-α, IL-6, IL-8, IL-12, IL-18, IL-17A, IFN-γ and IP-10. There were no differences in the cytokine profiles between patients and healthy individuals, except for increased IP-10 levels in RRMS (p=0.03) which is consistent with a previous study (Franciotta, D., et al., J. Neuroimmunol. 115, 192-198 (2001)). Thus, no apparent difference at the level of maturation or cytokine production could easily explain the source of Teff resistance. The cohorts were next used to examine T cell intrinsic factors known to be implicated in the development of Teff resistance.

Increased IL-6 mediated pSTAT3 correlates with impaired suppression of RRMS CD4⁺ T cells. The IL-6 pathway has been linked to pro-inflammatory immune responses and its relevance in driving Teff cell resistance has been demonstrated in both mice and humans (Korn, T., et al., Nat. Med. 13, 423-431 (2007); Goodman, W. A., et al., J. Immunol. 186(6), 3336-3345 (2011); Goodman, W. A., et al., J. Immunol. 183, 3170-3176 (2009)). Alterations in basal pSTAT3 and a genetic variant in STAT3 have been associated with RRMS. To address whether increased basal or enhanced signaling by IL-6 plays a role in the development of Teff resistance in MS, pSTAT3 was examined by flow cytometry at baseline and upon stimulation with IL-6 in a group of MS subjects and compared to controls.

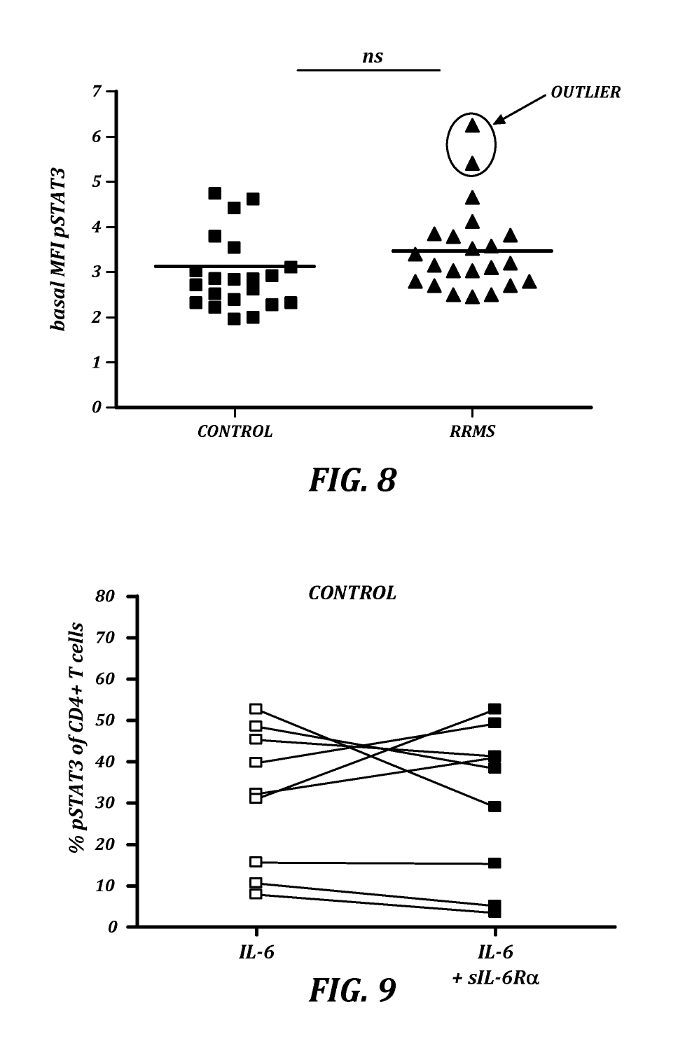

FIG. 8 shows similar levels of basal pSTAT3 between RRMS and control individuals. PBMC were thawed and basal MFI pSTAT3 expression was determined by gating on total CD4⁺ T cells for control and RRMS subjects in the first and second study cohort. Bars show means and p-values were determined by using an independent Student t-test. Two RRMS subjects in the first cohort had elevated baseline levels for MFI pSTAT3 (cut off MFI<5) and were considered as RRMS “outliers.” Unlike previous studies, there was no difference in basal pSTAT3 in CD4⁺ T cells between controls and RRMS subjects with the exception of two RRMS subjects that had markedly elevated baseline levels for MFI pSTAT3.

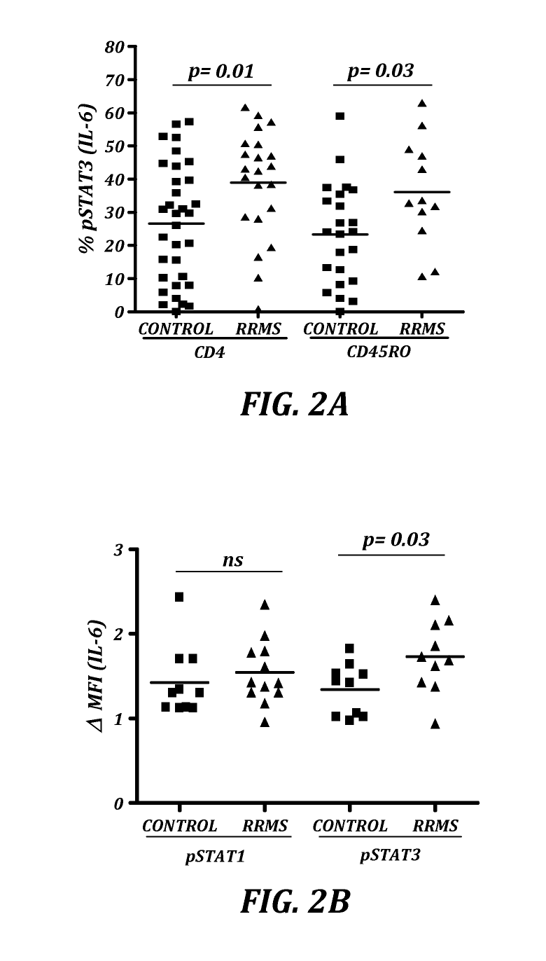

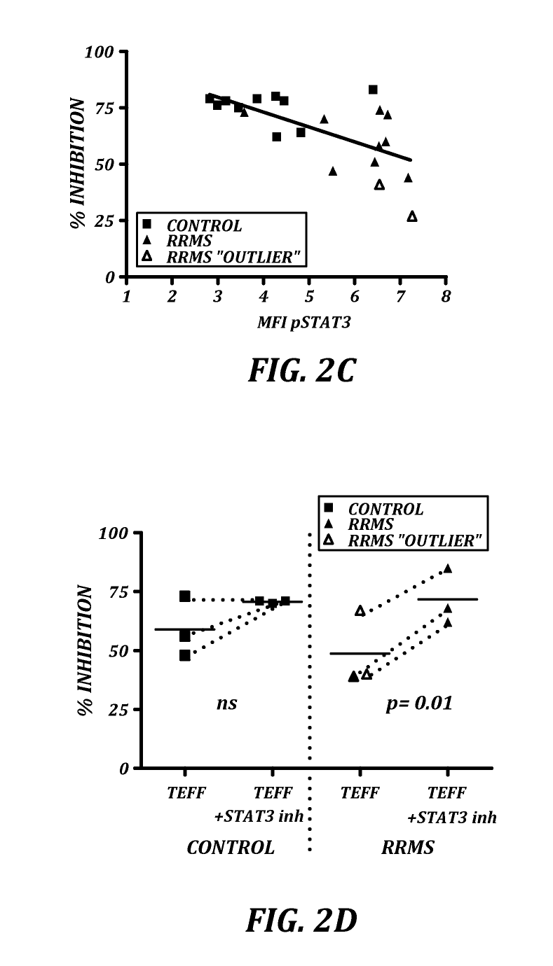

The relative percentage of pSTAT3 positive cells after exposure to IL-6 was significantly increased in the total CD4⁺ T cells as well as in the CD4⁺CD45RO⁺ memory T cell pool of RRMS subjects. FIG. 2 shows impaired suppression correlates with increased pSTAT3 in RRMS and can be reversed in the presence of a STAT3 inhibitor. Referring to FIG. 2A, PBMC from control and RRMS subjects of both cohorts were stimulated with 5 ng/ml IL-6, then analyzed by using an independent Student t-test. (see also FIG. 5B). In FIG. 2B, subjects from the first cohort were stimulated with IL-6, and ΔMFI was determined. Referring to FIG. 2B, in the first patient cohort, when impaired suppression was more pronounced than in the second cohort, the mean fold change for MFI pSTAT3 was also significantly increased in response to IL-6.

As noted above, FIG. 5B relates to a gating strategy for the calculation of percent inhibition in a suppression assay and a cytometric analysis of phosphorylated STAT3 and IL-6Rα surface expression. Referring to FIG. 5B, previously frozen PBMC were thawed and stimulated with 5ng/m1 IL-6 (blue line) which was followed by fixation and permeabilization prior to staining with anti-pSTAT3 and CD4. The histogram illustrates CD4⁺ T cells which were gated on live and the level of phosphorylated STAT3 was determined based on unstimulated PBMC (red line).

When the relationship between percent inhibition and MFI pSTAT3 expression in response to IL-6 for the subjects in the first cohort was examined, there was a significant negative correlation with lower suppression linked to an increase in pSTAT3 expression in RRMS subjects and controls (p=0.001; Spearman r=−0.6673). Referring to FIG. 2C, the percent inhibition for control and RRMS responder cells at a ratio of 1:4 was plotted against MFI pSTAT3 in response to IL-6. A correlation was determined for all subjects in the first cohort including the two RRMS “outliers” (Spearman r=−0.6673, p=0.001). Most notably, this finding showed a correlation between enhanced pSTAT3 signaling and impaired suppression in RRMS subjects, whereas CD4⁺ T cells from controls were found within a normal range of percent inhibition and have lower MFI pSTAT3 levels in response to IL-6.

IL-6 mediated Teff resistance can be reversed through the use of a STAT3 inhibitor (Goodman, W. A., et al., J. Immunol. 186(6), 3336-3345 (2011)). To determine if there was a mechanistic link between the observation of Teff resistance in RRMS and enhanced pSTAT3 in response to IL-6, samples from three subjects who had previously demonstrated Teff resistance and who continued off therapy were obtained. Suppression assays were performed in the presence of the STAT3 inhibitor using the same source of control Treg for RRMS and control Teff cells. In FIG. 2D, the percent inhibition for controls (n=3) and RRMS (n=3) subjects in the presence or absence of a STAT3 inhibitor, STATTIC V, was compared using a paired Student t-test. As shown in FIG. 2D, there was enhanced suppression in the presence of the STAT3 inhibitor for the control subjects, but a more marked increase in suppression for the RRMS samples (p<0.01), indicating that enhanced pSTAT3 in RRMS subjects may be one mechanism by which Teff cells escape suppression by Treg.

Thus, in one aspect, the use of a compound effective to reduce phosphorylation of STAT3 in the manufacture of a medicament for use in the treatment of multiple sclerosis in a subject in need thereof is provided.

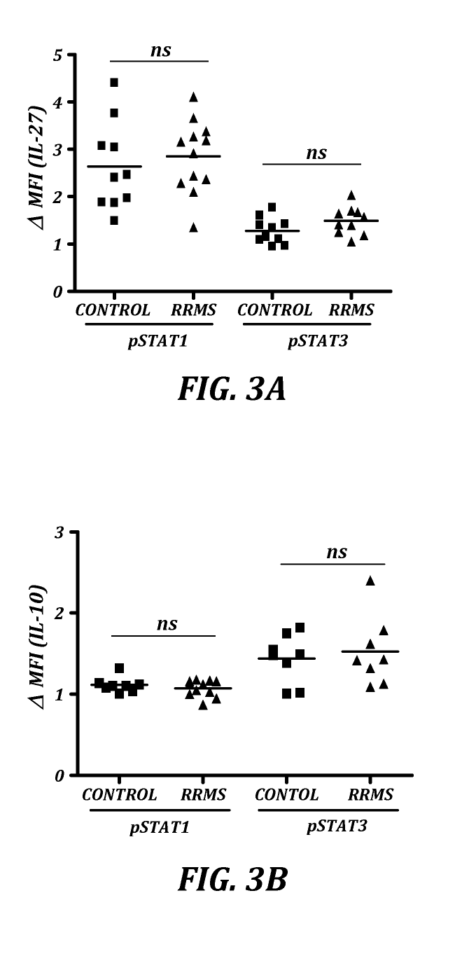

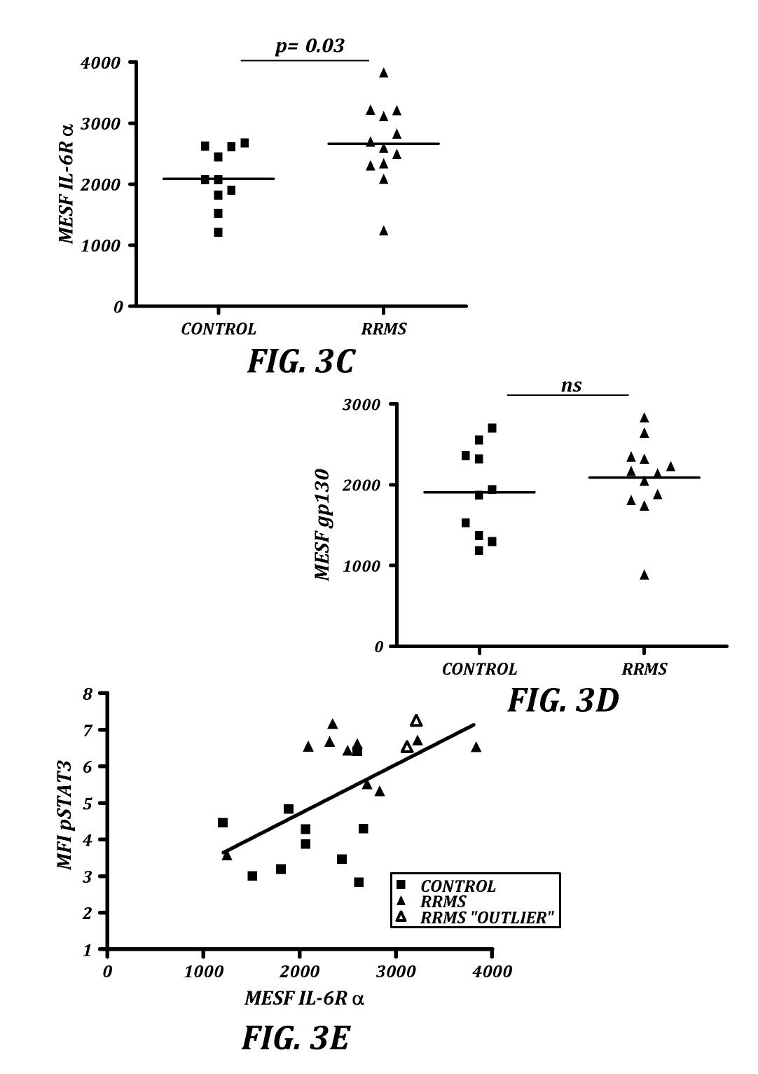

Enhanced pSTAT3 in response to IL-6 is associated with increased IL-6Rα expression on CD4⁺ T cells of RRMS subjects. To determine whether an increase in pSTAT3 is unique to IL-6 mediated signaling, the response to IL-27 and IL-10 which also signal through STAT3 but which utilized different cell surface receptors was examined. FIG. 3 shows IL-6Rα expression is enhanced on CD4⁺ T cells in RRMS. PBMC of control and RRMS subjects from the first cohort were assessed for CD4, pSTAT1 and pSTAT3 after activation with IL-27 (5 ng/ml) for 20 min (FIG. 3A) and with IL-10 (5 ng/ml) for 20 min (FIG. 3B). Two RRMS subjects were excluded for this analysis because their basal MFI pSTAT3 was above the cut off (basal MFI pSTAT3<5; see FIG. 8). The response to these cytokines as measured by fold change MFI pSTAT3 was no different in RRMS and control CD4⁺ T cells.

Exposure of CD4⁺ T cells to IL-6, IL-27 and IL-10 induces the formation of STAT1/STAT3 homo- and heterodimers. IL-6 preferentially activates STAT3 over STAT1 as does IL-10, while IL-27 stimulation leads to accentuated activation of STAT1 which favors regulation of inflammation. There was no shift in ΔMFI pSTAT3 relative to ΔMFI pSTAT1 in CD4⁺ T cells of RRMS subjects as compared to controls in response to IL-6, IL-27 or IL-10. Although increased levels of pSTAT3 in response to IL-6 were accompanied by increased pSTAT1 expression in RRMS subjects, it did not reach statistical significance when compared with controls. Taken together, these results indicate that the CD4⁺ T cells of RRMS subjects display enhanced pSTAT3 that is specific for IL-6, indicating that the mechanisms by which this occurs is likely related to the IL-6 receptor itself.

IL-6 signaling can be mediated via cell surface IL-6Rα-dependent classical signaling or via trans-signaling through the interaction of sIL-6Rα, IL-6 and cell surface gp130. To determine whether trans-signaling contributed to the differences in responsiveness to IL-6 in the in vitro assay, it was determined that the percent of pSTAT3 in CD4⁺ T cells was not significantly different in healthy individuals in the presence of either IL-6 or the combination of IL-6/sIL-6Rα, indicating that even at a high concentration of sIL-6Rα, IL-6 signaling is not further increased by trans-signaling in the in vitro culture system. FIG. 9 shows trans-signaling does not further enhance STAT3 phosphorylation in vitro. Referring to FIG. 9, PBMCs from controls were thawed and stimulated with 5 ng/ml IL-6 for 20 min via the classical signaling pathway or with the combination of 5 ng/ml IL-6 and 25 ng/ml sIL-6Rα for trans-signaling prior to fixation and staining with CD4⁺ and pSTAT3. Data was analyzed by gating on percent pSTAT3 of live CD4⁺ T cells and using a paired analysis.

These results indicated that alterations in the IL-6R signaling were likely due to alterations in the level or function of the cell surface receptor in the assay system. To directly test this, the expression levels of the components of the IL-6R including IL-6Rα (FIG. 3C) and gp130 (FIG. 3D) on the CD4⁺ T cells of RRMS and control subjects were compared. The level of IL-6Rα expression and gp130 on CD4⁺ T cells from control and RRMS subjects in the first cohort was determined by flow cytometry using Quantum R-PE MESF beads. Data was analyzed by using an independent Student t-test. There was a significant increase in the IL-6Rα levels on RRMS CD4⁺ T cells, but not for the signal transducing component gp130 (see also FIG. 5C). As noted above, FIG. 5C relates to gating strategy for calculation of percent inhibition in a suppression assay and a cytometric analysis of phosphorylated STAT3 and IL-6Rα surface expression. FIG. 5C shows PBMC stained in parallel for surface receptor components. CD4⁺ T cells were gated on live cells and the histogram shows the level of IL-6Rα expression (blue line) based on an isotype control (red line).

Nor was there any differences in the ratio of gp130/IL-6Rα on CD4⁺ T cells of control subjects and RRMS patients, because increased IL-6Rα was accompanied by increased gp130 expression in RRMS, but this increase did not reach statistical significance similar to what has been seen for pSTAT1 and pSTAT3 expression levels in response to IL-6 in RRMS individuals. In FIG. 3E, statistical analysis was performed to assess the relationship between IL-6Rα expression and MFI pSTAT3 levels in response to IL-6 stimulation. The line represents the correlation of control and RRMS data for the first cohort including the two RRMS “outliers” (Spearman r=0.5042, p=0.02). The role of increased IL-6Rα expression as a mechanism that leads to the enhancement of pSTAT3 in response to IL-6 in RRMS CD4⁺ T cells was further supported by the positive correlation between the density of IL-6Rα expression with MFI pSTAT3. This was also significant when the correlation was performed using the fold change MFI pSTAT3 in response to IL-6 (p=0.02; Spearman r=0.4925). These findings suggest that alterations in responsiveness to the pro-inflammatory cytokine IL-6 are present in RRMS subjects, and that this is in part due to an increased level of expression of the IL-6Rα on CD4⁺ T cells of these subjects.

Teff cell resistance is associated with active disease and increased pSTAT3 upon IL-6 stimulation. Although the findings show statistically significant differences between control and MS cohorts (e.g., see FIGS. 1A and 1B), the resistance to suppression was not a uniform characteristic of the RRMS subjects. Five out of fourteen (36%) of the subjects display normal levels of inhibition, while the percent inhibition for nine out of fourteen (64%) of the RRMS subjects was more than two standard deviations below the mean of controls. The clinical features that can differentiate these two groups were considered, and there was no correlation with gender or years since diagnosis. The difference between controls in the first and second cohorts is not statistically significant (p-value=0.0845). To account for the potential confounding effect of age on percent inhibition, several linear mixed models were constructed (cohort 1 only, cohort 2 only, and combined data set) where percent inhibition is a fixed effect and age a random effect. There was a weak correlation between percent inhibition and age in cohort 1 (R²˜0.56), however the contribution of age is only ˜0.2% of total residual variance (not significant). The F-test p-value for the fixed effect, percent inhibition, between control and RRMS in cohort 1 is less than 0.0001, adjusted for age as a random effect. No correlation between percent inhibition and age was found in cohort 2 and the difference in percent inhibition between control and RRMS was also statistically significant (p-value=0.026). When this analysis was extended to both cohorts in combination the difference in percent inhibition between control and RRMS remained significant at p-value<0.0003 level.

Nonetheless, disease activity did differentiate the two groups. Disease activity was based on clinical exacerbations or the presence of gadolinium enhancing lesions on MRI. “Active” disease was defined as two or more clinical exacerbations or presence of one or more gadolinium enhancing lesions on MR imaging within two years of sampling, while all other individuals were classified as “quiescent disease” (e.g., see Table 1 and 2). The IL-6 levels were found to be similar in the serum of active and inactive RRMS patients.

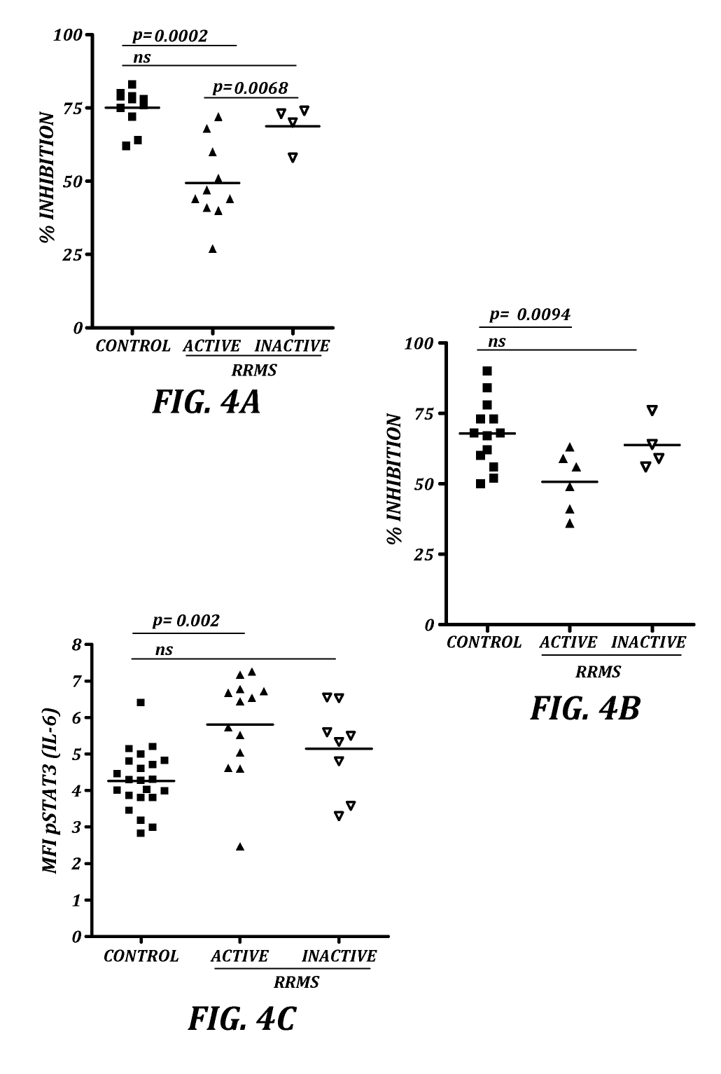

FIG. 4 shows that resistance to suppression in active RRMS correlates with increased levels of pSTAT3. As shown in FIG. 4A and 4B, control aTreg were co-cultured at a ratio of 1:4 with either Teff from control subjects or subjects with RRMS, as described above in relation to FIG. 1, which then were divided into active RRMS and inactive RRMS subjects. While subjects with inactive disease from the first cohort did not show a significant impairment in suppression as compared to controls, those with active disease had a significantly lower level of inhibition from control and inactive RRMS subjects and comprised the majority of RRMS patients with impaired suppression (p=0.0002, FIG. 4A; see also Table 1). A positive correlation between impaired suppression and disease activity was confirmed in the second RRMS cohort as well (p=0.009) (FIG. 4B; see also Table 2). Referring to FIG. 4C, PBMC from control subjects, subjects with active RRMS, and subjects with inactive RRMS were stimulated with IL-6 for 20 min and analyzed by using an independent Student t-test. As shown in FIG. 4C, when both cohorts were classified into active and inactive disease, the MFI pSTAT3 levels were found to be significantly increased in subjects with active disease (p<0.002). These findings indicate that enhanced IL-6 mediated phosphorylation of STAT3 contributes to impaired regulation in RRMS and is prominent in patients with active disease.

Thus, in one aspect, a method for characterizing relapsing-remitting multiple sclerosis in a subject is described. The method comprises the steps of (a) obtaining a sample comprising CD4⁺ T cells from a subject having multiple sclerosis; (b) exposing at least a portion of CD4⁺ T cells from the sample to IL-6 to provide stimulated CD4⁺ T cells; (c) quantifying an amount of pSTAT3 in the stimulated CD4⁺ T cells; and (d) determining if the amount of pSTAT3 is increased, wherein increased pSTAT3 is indicative of an active phase of relapsing-remitting multiple sclerosis in a subject. Loss of tolerance due to Teff cell resistance has been demonstrated in multiple autoimmune diseases. In SLE, the Teff of patients with active disease are less sensitive to the suppressive function than those from inactive SLE patients (Venigalla, R. K., et al., Arthritis Rheum. 58, 2120-2130 (2008)). In RA and JIA T cells isolated from the synovial fluid are more resistant to suppression by Treg than those isolated from the peripheral blood Wehrens, E. J., et al., Blood 118(13), 3538-3548 (2011); van Amelsfort, J. M., et al., Arthritis Rheumat. 50, 2775-2785 (2004)) and this is linked to the presence of IL-6 and TNFα in the synovial fluid (Herrath, J., et al., Eur. J. Immunol. 41, 2279-2290 (2011)). The T cells in psoriatic skin are resistant to Treg mediated suppression through an IL-6 mediated mechanism (Goodman, W. A., et al., J. Immunol. 183, 3170-3176 (2009)). Several groups have demonstrated Teff cell resistance in T1D subjects (Lawson, J. M., et al., Clin. Exp. Immunol. 154, 353-359 (2008); Schneider, A., et al., J. Immunol. 181, 7350-7355 (2008)), which in the studies appears to be a T cell intrinsic defect. Together these studies indicate that disease activity, local cytokine milieu and T cell intrinsic factors can play a role in the acquisition of Teff resistance in autoimmunity.

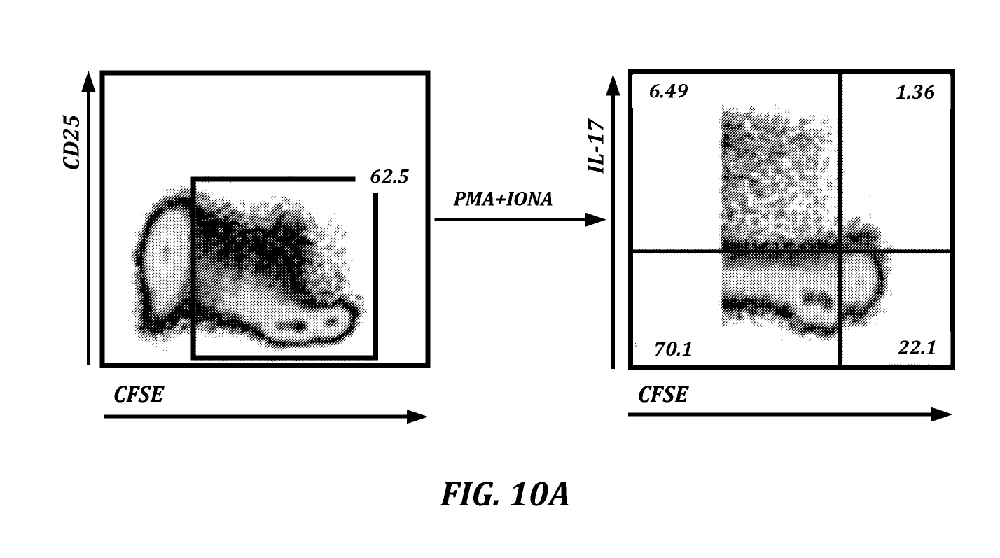

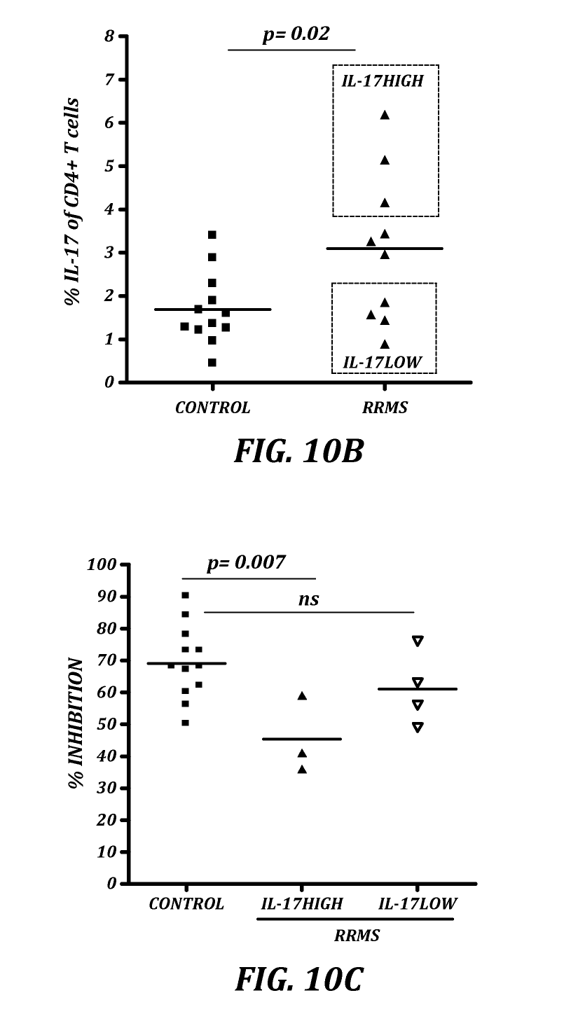

Prior to this report Teff resistance in MS had only been addressed in a very limited number of subjects (Viglietta, V., et al., J. Exp. Med. 199, 971-979 (2004)). The present study addressed this question in a larger RRMS cohort (n=14) and was confirmed in a second cohort of untreated RRMS patients (n=10). Studies of Teff resistance require the use of allogeneic control Treg, and the source and type of Treg differs between studies. In most cases Treg are isolated directly from the peripheral blood of healthy donors based on cell surface markers; alternatively nTreg can be isolated then expanded in vitro prior to their use (Putnam, A. L., et al., Diabetes 58, 652-662 (2009)), or Treg can be induced in vitro (aTreg) and used in these assays. An effector T cells ability to evade suppression may be influenced by the source of Treg, in previous studies there were no difference between directly isolated nTreg and in vitro generated a aTreg with respect to suppression and Teff resistance in T1D (Schneider, A., et al., J. Immunol. 181, 7350-7355 (2008)). In the current study additional assays were performed using expanded natural Tregs as a source of Treg and again found a similar level of suppressive function in RRMS and control subjects. Given these caveats, the results indicate that Teff cell resistance is present in a subgroup of active RRMS patients raising the question as to the mechanisms that underlie this defect in regulation. Lineage and maturation state of CD4⁺ T cells can influence the susceptibility to suppression via Treg. In mice and man, cell intrinsic resistance is linked to the CD4⁺CD45RO⁺ memory T cell compartment (Yang, J., et al., Proc. Natl. Acad. Sci. USA 104, 19954-19959 (2007)) and within the memory compartment, Th17 cells have also been shown to be resistant to suppression (Korn, T., et al., Annu. Rev. Immunol. 27, 485-517 (2009)). There was not a significant difference in the percentage of CD4⁺CD45RO⁺ cells among the RRMS CD4⁺CD25⁻ T cell pool used in the in vitro co-culture assay and controls. However, because IL-6 is essential for Th17 cell differentiation (Volpe, E., et al., Nat. Immunol. 9, 650-657 (2008)) and the subjects displayed enhanced responsiveness to IL-6, the composition of the memory compartment needed to be addressed. To do this the intracellular IL-17 expression in CD4⁺ Teff cells that had divided in the co-culture assays was examined. FIGS. 10A, 10B, and 10C show IL-17 production is increased in RRMS and correlates with less suppression. FIG. 10A is a schematic model for a representative intracellular IL-17 staining: CFSE-labeled CD4⁺CD25⁻ T cells were co-cultured at a ratio of 1:4 (aTreg:Teff) in the presence of anti-CD3/anti-CD28 coated beads. On day four, the co-culture was restimulated with PMA+Ionomycin for five hours prior to fixation and staining with CD4, CD25 and intracellular IL-17A. Analysis was based on an unstimulated sample to determine percent IL-17 producing CD4⁺ T cells after gating out the Treg population.

Referring to FIG. 10B, control aTreg were co-cultured either in the presence of control or RRMS responder cells and activated with beads. On day four, co-cultures were restimulated and analyzed for percent IL-17 of CD4⁺ T cells after gating out the Treg population. RRMS subjects were grouped into IL-17^{high} and IL-17^{low} producers. Three out of ten RRMS subjects had a twofold greater percentage of IL-17 producing cells than the control subjects; however, these cells represent a minority of the proliferating cells in this assay (1 to 6% IL-17 producing CD4⁺ T cells).

In the majority of RRMS subjects a similar percentage of Th17 cells were found among the divided Teff population on day 4 as controls. Referring to FIG. 10C, co-cultures were analyzed for percent inhibition by CFSE dye dilution and assessed for percent IL-17 on CD4⁺ T cells. Bars show means for Control, RRMS IL-17^{high} and RRMS IL-17^{low} producers and p-values were determined by using an independent Student t-test. As shown in FIG. 10C, a defect in Treg-mediated regulation was seen in RRMS CD4⁺ IL-17^{high} producers when compared with healthy individuals and these results are in line with other studies, however, the percentage of IL-17 producing Teff did not correlate with disease activity. Thus, this can represent an additional mechanism driving impaired Treg-mediated suppression in RRMS patients, however, it is not found in the majority of subjects nor does it explain the increase in Teff resistance seen with active disease.

A role for IL-6 mediated signaling in the development of Teff resistance in RRMS is supported by the resistance of IL-6 and STAT3 deficient mice to EAE induction (Samoilova, E. B., et al., J. Immunol. 161, 6480-6486 (1998); Liu, X., et al., J. Immunol. 180, 6070-6076 (2008)). With respect to Teff cell resistance, Pasare et al. have shown that IL-6 allows effector T cells to overcome Treg-mediated regulation (Pasare, C., and Medzhitov, R., Science 299, 1033-1036 (2003)) and a recent study by Goodman et al. in healthy donors has demonstrated IL-6 mediated Teff resistance which could be reversed in the presence of a STAT3 inhibitor (Goodman, W. A., et al., J. Immunol. 186(6), 3336-3345 (2011)). Increased levels of IL-6 leading to Teff resistance is also supported by the findings of Teff resistance in psoriatic skin where IL-6 is elevated, and in synovial fluid of RA and JIA subjects where Teff resistance can be reversed by inhibition of IL-6 (Goodman, W. A., et al., J. Immunol. 183, 3170-3176 (2009); Herrath, J., et al., Eur. J. Immunol. 41, 2279-2290 (2011)).

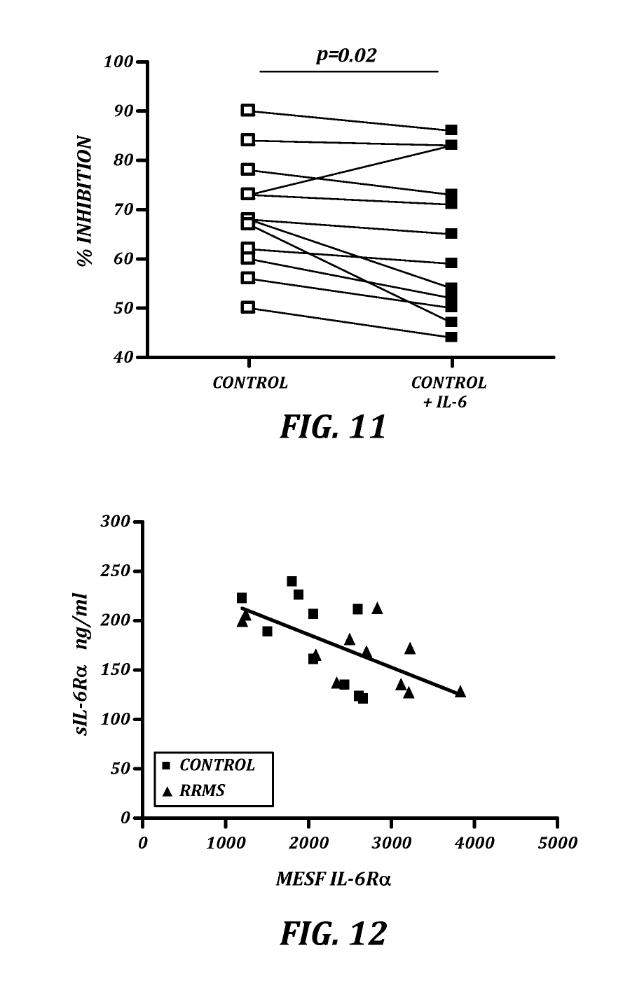

To determine whether IL-6R mediated signaling could alter suppression in the assay system, Teff were cultured from healthy subjects in the presence or absence of IL-6 in the in vitro co-culture system and assessed percent inhibition on day 4. FIG. 11 shows IL-6 impairs Treg-mediated regulation in healthy individuals. Referring to FIG. 11, CFSE-labeled CD4⁺CD25⁻ T cells were cultured at a ratio of 1:4 (control aTreg:control Teff) either in the absence or presence of 50 ng/ml IL-6 and activated with anti-CD3/anti-CD28 coated beads. FACS analysis was performed to determine percent inhibition on day 4 and data is represented by using a paired analysis (Correlation coefficient (r) 0.8934). Proliferation was unaffected in cultures with Teff alone, but suppression by Treg was significantly decreased in the presence of IL-6 (p=0.02).

Although IL-6 was detected in the co-culture supernatant, it was not possible to detect increases in IL-6 production in the RRMS subjects that would explain the resistance to suppression that was seen indicting that the source of Teff resistance in the subjects was not directly due to increase IL-6 in the co-culture, but could be due to enhanced responsiveness of the RRMS Teff cells to IL-6.

Phosphorylation of STAT3 has been linked to Teff cell resistance through several mechanisms; strong TCR-dependent stimulation via anti-CD3 and anti-CD28 Abs results in high levels of pSTAT3 and resistant Teff (Goodman, W. A., et al., J. Immunol. 186(6), 3336-3345 (2011); Putnam, A. L., et al., J. Autoimmun. 24, 55-62 (2005)). IL-21 and IL-6, both of which signal via STAT3, have been shown to promote Teff resistance, which in the case of IL-6 can be reversed by the use of STAT3 inhibitor. In RRMS, pSTAT3 levels are elevated in patients at the time of relapse (Jakkula, E., et al., Am. J. Hum. Genet. 86, 285-291 (2010); Frisullo, G., et al., J. Neurosci. Res. 84, 1027-1036 (2006)) and recent GWAS have confirmed that variants in the STAT3 locus are among other genes that are linked to MS. There was no significant increase in basal pSTAT3 or total STAT3 in the RRMS subjects with the exception of two RRMS individuals had elevated basal pSTAT3 levels (see, e.g., FIG. 8) which could have been the result of a recent exposure to IL-6 in vivo or genetic factors that were unique to these subjects. The observation that the enhanced level of pSTAT3 in RRMS subjects was unique to IL-6 and not seen in response to IL-21, IL-10 and IL-27, all of which signal via STAT3, indicates that the dominant mechanisms of enhanced pSTAT3 in these patients is directly linked to IL-6R signaling. This is further supported by the significant increase in the expression of IL-6Rα on RRMS CD4⁺ T cells and the positive correlation between pSTAT3 in response to IL-6 and the expression of IL-6Rα.

IL-6Rα expression on the cell surface is regulated at the level of transcription by IL-1α, IFN-γ and IL-6 itself and results in upregulated IL-6R mRNA levels (Sanceau, J., et al., J. Immunol. 147, 2630-2637 (1991); Takizawa, H., et al., Am. J. Physiol 270, L346-L352 (1996)). Further, IL-6Rα expression is decreased upon TCR activation due to cleavage of the membrane bound form to sIL-6Rα by the metalloproteinase ADAM17. When control CD4⁺ T cells were co-cultured with RRMS serum, IL-6Rα expression was not altered compared to culture conditions with control serum in the presence or absence of anti-CD3/anti-CD28 bead stimulation. However, when the level of sIL6Rα in the serum of the subjects was examined, a lower concentration of sIL-6Rα was found in RRMS subjects compared to controls. FIG. 12 shows sIL-6Rα negatively correlates with IL-6Rα expression. sIL-6Rα levels in the serum of cohort 1 were determined by ELISA and the line represents correlation with IL-6Rα surface expression (Spearman r=−0.5701, p=0.007). This decrease in sIL-6Rα negatively correlated with cell surface IL-6Rα expression, indicating that there may be a link between the enhanced response to IL-6 that was observed in RRMS subjects and the release of membrane bound IL-6Rα to form sIL-6Rα.

Surprisingly, Teff resistance is present in only a subset of RRMS patients. A criterion for entry into the study was that the RRMS subjects were not on immune modulating therapy, in order to avoid the added complexity of a drug effect on T cell suppression. By choosing subjects in this way, it was determined that a subset of individuals studied had relatively mild or inactive disease while others had disease that was active. The majority of the RRMS subjects with active disease were not flaring at the time of the samples collection, so Teff resistance and enhanced pSTAT3 in response to IL-6 are not a consequence of disease flares, but instead may be a feature of active or aggressive disease in RRMS subjects. In these individuals, IL-6 produced by microglia, astrocytes, endothelial cells, neurons, oligodendrocytes, or infiltrating T cells (Gruol, D. L., and Nelson, T. E., Mol. Neurobiol. 15, 307-339 (1997)) results in enhanced pSTAT3 and resistance of the pathogenic CD4⁺ T cells to regulation mediated by regulatory T cells of the CNS. These findings show that IL-6Rα and pSTAT3 in response to IL-6 can be used as a biomarker to predict disease activity or responsiveness to immunomodulatory therapies that target the IL-6/IL-6Rα/STAT3 pathway such as Tocilizumab, an IL-6Rα antagonist.

Thus, in one aspect, a method for predicting autoimmune or demyelinating disease progression in a subject is provided. The method includes (a) determining a level of IL-6 responsiveness of CD4⁺ T cells from a subject; and (b) predicting progression of a disease or condition in the subject based upon the level of IL-6 responsiveness.

The determination of clinical prognosis in RRMS subjects was based on the assessment of CD4⁺ T cell suppression by Treg, the level of IL-6Rα expression and IL-6 responsiveness as measured by pSTAT3 in CD4⁺ T cells by flow cytometric analysis and measurement of serum sIL-6Rα levels. There are differences in the responsiveness of CD4⁺ T cells from individuals with relapsing remitting multiple sclerosis (RRMS) to the cytokine IL-6 as measured by the phosphorylation of STAT3 and the cell surface expression of IL-6Rα by flow cytometry. Differences between RRMS subjects with respect to these measures correlate with at least two features of subjects with RRMS, for example, their clinical status and the immunologic profile of effector T cell response to suppression by regulatory T cells (Treg).

Thus, in one aspect, a method for identifying an autoimmune or demyelinating disease in a subject is provided. The method comprises the steps of (a) obtaining a sample comprising CD4⁺ T cells from a subject; (b) determining a level of IL-6 responsiveness of CD4⁺ T cells from the sample; (c) comparing the level of IL-6 responsiveness determined in step (b) with a level of IL-6 responsiveness of CD4⁺ T cells in a sample from a healthy subject; and (d) identifying the subject as having an autoimmune or demyelinating disease based upon an elevated level of IL-6 responsiveness in the subject. In one embodiment, the level of IL-6 responsiveness is determined by a level of IL-6Rα and/or a level of pSTAT3 expression. In one embodiment, the subject is identified as having multiple sclerosis or relapsing remitting multiple sclerosis. In one embodiment, the subject has been diagnosed with or treated for multiple sclerosis.

In one embodiment, a course of treatment for a subject having an elevated level of IL-6 responsiveness is more aggressive than a course of treatment for a subject not having an elevated level of IL-6 responsiveness. Standard methods of treatment for multiple sclerosis include treatments aimed at modifying the course of progression, treating relapses and flare ups, and managing symptoms, among others. For example, the following agents can reduce disease activity and disease progression for many individuals with RRMS, including those with secondary progressive disease who continue to have relapses: beta interferons, teriflunomide, glariramer acetate, fingolimod, mitoxantrone, and natalizumab. Of these, mitoxantrone is often used for aggressive forms of RRMS or secondary progressive MS.

In another aspect, a method for predicting autoimmune or demyelinating disease progression in a subject is described. The method comprises the steps of (a) obtaining a sample comprising CD4⁺ T cells from a subject; (b) determining a level of IL-6 responsiveness of CD4⁺ T cells from the sample; (c) comparing the level of IL-6 responsiveness determined in step (b) with a level of IL-6 responsiveness of CD4⁺ T cells in a sample from a healthy subject; and (d) predicting progression of a disease or condition in the subject based upon an elevated level of IL-6 responsiveness in the subject. In one embodiment, the level of IL-6 responsiveness is determined by a level of IL-6Rα and/or pSTAT3 expression. In one embodiment, the disease or condition is multiple sclerosis or relapsing-remitting multiple sclerosis. In one embodiment, the subject has been diagnosed with or treated for multiple sclerosis. In one embodiment, a course of treatment for a subject having an elevated level of IL-6 responsiveness is more aggressive than a course of treatment for a subject not having an elevated level of IL-6 responsiveness.

RRMS patients can be categorized as those with “active” or “quiescent” disease. “Active” patients are defined by two or more relapses or Gd+ lesions on routine imaging within the last two years, or Gd+ lesions. This characterization of RRMS has been used in several pivotal Phase II trials of experimental therapeutics in MS, where entry criteria to define “active disease ” are some combination of the following: two relapses in the two years prior and/or one relapse in the year prior to enrollment; or presence of Gd+ lesions in the year prior to enrollment. (For example, see clinical trial identifier nos. NCT00109161, NCT01064401, NCT00530348, NCT01116427, and NCT01081782, available at ClinicalTrials.gov, a service of the U.S. National Institutes of Health.) This approach allows for the identification of markers/characteristics in populations that might serve to identify individuals who are at higher risk of exhibiting active disease versus those who might be expected to have a more benign clinical course. Such predictive markers could be used to assist patients and clinicians in determining whether and which (more aggressive) therapies, for example mitoxantrone therapy, increased dosage amounts or frequency of standard therapies, might be appropriate.

An exacerbation of MS is caused by inflammation in the central nervous system that causes damage to the myelin and slows or blocks the transmission of nerve impulses. Exacerbations can be mild or severe enough to interfere with a person's ability to function at home and at work. Severe exacerbations are most commonly treated with high-dose corticosteroids or plasma exchange.

Thus, in one aspect, a method for monitoring autoimmune or demyelinating disease progression in a subject is described. The method comprises the steps of (a) obtaining a first sample comprising CD4⁺ T cells from a subject; (b) determining a level of IL-6 responsiveness of CD4⁺ T cells from the first sample; (c) obtaining a second sample comprising CD4⁺ T cells from a subject; (d) determining a level of IL-6 responsiveness of CD4⁺ T cells from the second sample; (e) comparing the level of IL-6 responsiveness of CD4⁺ T cells from the first sample and the second sample; and (f) identifying disease progression in the subject, wherein a change in the level of IL-6 responsiveness is indicative of disease progression. In one embodiment, the level of IL-6 responsiveness is determined by a level of IL-6Rα and/or pSTAT3 expression. In one embodiment, an increase in the level of IL-6 responsiveness is indicative of the subject having multiple sclerosis or relapsing-remitting multiple sclerosis. In one embodiment, the subject has been diagnosed with or treated for multiple sclerosis. In one embodiment, a course of treatment for a subject having an increased level of IL-6 responsiveness is more aggressive than a course of treatment for a subject not having an elevated level of IL-6 responsiveness.

The Teff cells of individuals with active RRMS demonstrate a resistance to Treg mediated suppression of proliferation as measured in vitro. This resistance to suppression correlates with an enhanced response to IL-6 as measured by pSTAT3 after exposure to IL-6 (in vitro 20 minutes). Further, the enhanced response to IL-6 correlates with the level of expression of IL-6Rα on the surface of CD4⁺ T cells. Additional studies have shown that an examination of soluble sIL-6Rα in the serum of RRMS patients demonstrates a negative correlation with IL-6Rα cell surface expression.

These findings demonstrate that measurements of Teff resistance in vitro, flow cytometric analysis of CD4⁺ T cells for IL-6Rα cell surface expression, and pSTAT3 in response to IL-6 and serum sIL-6Rα are useful biomarkers with the capability to assist in the prediction of future disease activity as well as the selection of immunotherapies to target this immunologic and biochemical pathways in RRMS.

Thus, in one aspect, a method for treating multiple sclerosis comprising a composition selected from the group consisting of (a) an IL-6 inhibitor; (b) an IL-6Rα antagonist; (c) a sIL-6Rα antagonist; and/or (d) a STAT3 phosphorylation blocker or inhibitor, wherein the composition is administered for a time and in an amount effective to treat and/or ameliorate the symptoms of multiple sclerosis.

### EXAMPLES

The following example merely illustrates the best mode now contemplated for practicing the claimed methods and/or producing the disclosed compositions, but should not be construed to limit the disclosure.

### Example 1

This example describes subject selection, antibodies and reagents, Phosflow™ cytometric analysis, intracellular cytokine staining and a color coded microsphere based analysis (Luminex® analysis), CFSE-based (5,6-carboxy fluorescein diacetate succinimidyl ester-based) polyclonal suppression assays, and statistical analysis.

Subjects

Fresh blood samples and frozen peripheral blood mononuclear cells (PBMCs) were obtained from participants in the Benaroya Research Institute Immune Mediated Disease Registry (IMD) and the JDRF-BRI Center for Translational Research at Virginia Mason Research Center. Two patient cohorts and healthy controls were selected for these studies. A diagnosis of RRMS (relaxing remitting multiple sclerosis) was based on Revised McDonald Diagnostic Criteria for MS (Polman, C. H., et al., Ann. Neurol. 58, 840-846 (2005)). Disease activity was based on presence of clinical exacerbations or gadolinium enhancing lesions on magnetic resonance imaging (MRI). Subjects were defined as exhibiting “active” or “quiescent” disease based on criteria used in clinical trials. “Active” disease was defined as two or more clinical exacerbations or presence of one or more gadolinium enhancing lesions on MRI within two years of sampling, while all other individuals were classified as “quiescent disease” (Tables 1 and 2, supra). All subjects were off immune modulating and immunosuppressive therapies at the time of study and for at least three months prior to the blood draw. Control subjects were recruited from the BRI IMD registry, and were selected due to a lack of autoimmune disease, or any family history of autoimmunity. The research protocols were approved by the Institutional Review Board (IRB) at Benaroya Research Institute.

Antibodies and Reagents

Antibodies for flow cytometric analysis and sorting include: anti-CD4, -CD25, -CD45RO, -CD130, -CD126 (BD Pharmingen, San Jose, Calif.); and -FOXP3 (clone: 206D) with its IgG₁control (Biolegend, San Diego, Calif.). Antibodies and reagents for Phosflow™ cytometric analysis include: mouse anti-STAT3 (pY705), mouse anti-STAT1 (pY701), cell fixation buffer and cell permeation buffer (Fix Buffer I and Perm Buffer III, BD Pharmingen). Phosflow™-specific activation was induced by recombinant human IL-6, IL-27 and IL-10 from BD Pharmingen. Quantum™ MESF (molecules of equivalent soluble fluorochrome microbead) kits for quantitation of fluorescence intensity were purchased from Bangs Laboratories, Inc. (Fishers, Ind.). Fluorescent labeled antibodies for intracellular cytokine (IC) staining include Alexa Fluor® 700 anti-human CD4⁺ and Alexa Fluor® 647 anti-human IL-17A (Biolegend). Activation for Treg induction cultures was performed with purified anti-CD3 (UCHT1) and anti-CD28 (CD28.2) from BD Pharmingen.

Phosflow™ Cytometric Analysis, Intracellular Cytokine Staining and Luminex® Analysis

BD Biosciences Phosflow™ staining was performed as per the manufacturer instructions (BD Biosciences, San Diego, Calif.). PBMCs were activated in 1% pooled human serum (PHS) (Omega Scientific, Inc., Tarzana, Calif.) media in the presence of IL-6, IL-27 or IL-10. Cells were fixed and permeabilized, prior to staining with anti-pSTAT1, pSTAT3, CD4⁺ and CD45RO. Data was acquired using FACS Calibur (BD Biosciences), then analyzed by using FlowJo (Tree Star, Ashland, Oreg.). Baseline mean fluorescence intensity (MFI) levels were based on unstimulated PBMC cultured in 1% PHS media. The fold change MFI of pSTAT1 and pSTAT3 was determined to normalize for changes in baseline MFI (geometric mean (MFI) of the stimulated population/geometric mean (MFI) of the unstimulated population). For IC cytokine analysis, cells were stimulated for five hours with Phorbol Myristate Acetate/Ionomycin (PMA/Iono) in the presence of a golgi protein transport inhibitor containing monensin (BD Golgi-Stop™) and Brefeldin A for the last four hours. Analysis was performed by flow cytometry (LSR II, BD Biosciences) and data was acquired using DIVA software (BD Biosciences).

Serum and tissue culture supernatant concentrations were determined using a cytokine specific bead based antibody assay (Bio-Plex™ Human Cytokine Group I 10-plex Assay (Bio-Rad, Hercules, Calif., and Luminex Corporation, Austin, Tex.) and human IL-6R Platinum ELISA kit (eBioscience, San Diego, Calif.) in accordance with the manufacturer's protocols for the induction and isolation of CD4⁺CD25⁺FOXP3⁺ Treg.

PBMC were isolated from human peripheral blood by centrifugation over Ficoll-Hypaque gradients. CD4⁺ T cells were isolated via negative selection using the CD4⁺ T cell isolation kit (Miltenyi Biotec, Auburn, Calif.). aTreg were induced as previously described (Schneider, A., et al., J. Immunol. 181, 7350-7355 (2008)) from the CD4⁺CD25⁻ T cells obtained from total CD4⁺ T cells by negative selection with Miltenyi CD25 microbeads. Briefly, CD4⁺CD25⁻ T cells were cultured in 24-well plates with irradiated autologous antigen presenting cells (APC) (5000 rad) at a ratio of 1:2, activated with soluble anti-CD3 (5 μg/ml) and maintained for ten days in media. IL-2 (200 IU/ml) was added on day six. aTreg from the induction culture were isolated by cell sorting of the 5% of T cells with the highest CD25 expression via a FACS Vantage (Becton Dickinson). The percent of FOXP3⁺ T cells among the CD4⁺CD25^{bright} cells ranged from 55% to 85%.

CFSE-Based Polyclonal Suppression Assays

CD4⁺CD25⁻ responder cells were isolated as described above via negative selection using Miltenyi microbeads. Allogeneic CD4⁺CD25⁻ T cells were carboxyfluorescein succinimidyl ester (CFSE)-labelled and cultured at a ratio of 1:4 with aTregs and Dynabeads® (M-450 CD3/CD28 T, Dynal Biotech A.S.A./Life Technologies Corporation, Grand Island, N.Y.) at a ratio of 1:5 (beads:responder cells) as described previously (Schneider, A., and Buckner, J. H., Methods Mol. Biol. 707, 233-241 (2011)). Where indicated, CD4⁺CD25⁻ T cells were incubated with a phosphorylation inhibitor of STAT3 (“STATTIC V,” Santa Cruz Biotechnology, Dallas, Tex.) at 200 ng/ml for one hour, then washed and cultured, as described above. Analysis was performed on day four by flow cytometry (FACS Calibur). Data was acquired using CellQuest™ Pro software (BD Bioscience) and analyzed by FlowJo (Tree Star).

Statistical Analysis

Statistical analyses reported in this paper include independent and paired Student t-tests with the Welch's correction, two-tailed nonparametric correlation (Spearman), linear regression and linear mixed modeling. Analyses were performed using PRISM with the exception of linear mixed models that were performed using SAS 9.3 & JMP Genomics 6.0 (SAS Institute Inc., Cary, N.C.). Values <0.05 were considered significant.

While illustrative embodiments have been illustrated and described, it will be appreciated that various changes can be made therein without departing from the spirit and scope of the invention.

The embodiments of the disclosure in which an exclusive property or privilege is claimed are defined as follows:

1. A method for identifying an autoimmune or demyelinating disease in a subject, comprising:
(a) obtaining a sample comprising CD4⁺ T cells from a subject;
(b) determining a level of IL-6 responsiveness of CD4⁺ T cells from the sample;
(c) comparing the level of IL-6 responsiveness determined in step (b) with a level of IL-6 responsiveness of CD4⁺ T cells in a sample from a healthy subject; and
(d) identifying the subject as having an autoimmune or demyelinating disease based upon an elevated level of IL-6 responsiveness in the subject.

2. The method of claim 1, wherein the level of IL-6 responsiveness is determined by a level of IL-6Rα and/or a level of pSTAT3 expression.

3. The method of claim 1 or 2, wherein the subject is identified as having multiple sclerosis or relapsing-remitting multiple sclerosis.

4. The method of claim 1 or 2, wherein the subject has been diagnosed with or treated for multiple sclerosis.

5. The method of claim 1 or 2, wherein a course of treatment for a subject having an elevated level of IL-6 responsiveness is more aggressive than a course of treatment for a subject not having an elevated level of IL-6 responsiveness.

6. A method for predicting autoimmune or demyelinating disease progression in a subject, comprising:
(a) obtaining a sample comprising CD4⁺ T cells from a subject;
(b) determining a level of IL-6 responsiveness of CD4⁺ T cells from the sample;
(c) comparing the level of IL-6 responsiveness determined in step (b) with a level of IL-6 responsiveness of CD4⁺ T cells in a sample from a healthy subject; and
(d) predicting progression of a disease or condition in the subject based upon an elevated level of IL-6 responsiveness in the subject.

7. The method of claim 6, wherein the level of IL-6 responsiveness is determined by a level of IL-6Rα and/or pSTAT3 expression.

8. The method of claim 6 or 7, wherein the disease or condition is multiple sclerosis or relapsing-remitting multiple sclerosis.

9. The method of claim 6 or 7, wherein the subject has been diagnosed with or treated for multiple sclerosis.

10. The method of claim 6 or 7, wherein a course of treatment for a subject having an elevated level of IL-6 responsiveness is more aggressive than a course of treatment for a subject not having an elevated level of IL-6 responsiveness.

11. A method for monitoring autoimmune or demyelinating disease progression in a subject, comprising:
(a) obtaining a first sample comprising CD4⁺ T cells from a subject;
(b) determining a level of IL-6 responsiveness of CD4⁺ T cells from the first sample;
(c) obtaining a second sample comprising CD4⁺ T cells from a subject;
(d) determining a level of IL-6 responsiveness of CD4⁺ T cells from the second sample;
(e) comparing the level of IL-6 responsiveness of CD4⁺ T cells from the first sample and the second sample; and
(f) identifying disease progression in the subject, wherein a change in the level of IL-6 responsiveness is indicative of disease progression.

12. The method of claim 11, wherein the level of IL-6 responsiveness is determined by a level of IL-6Rα and/or pSTAT3 expression.

13. The method of claim 11 or 12, wherein an increase in the level of IL-6 responsiveness is indicative of the subject having multiple sclerosis or relapsing-remitting multiple sclerosis.

14. The method of claim 11 or 12, wherein the subject has been diagnosed with or treated for multiple sclerosis.

15. The method of claim 11 or 12, wherein a course of treatment for a subject having an increased level of IL-6 responsiveness is more aggressive than a course of treatment for a subject not having an elevated level of IL-6 responsiveness.

16. A method for predicting disease activity or responsiveness to immunomodulatory therapies in a subject with an autoimmune or demyelinating disease, comprising:
(a) obtaining a sample comprising CD4⁺ T cells from a subject;
(b) exposing at least a portion of CD4⁺ T cells from the sample to IL-6 to provide stimulated CD4⁺ T cells;
(c) quantifying an expression level of a biomarker in the stimulated CD4⁺ T cells; and
(d) determining if the subject is in an active phase of the autoimmune or demyelinating disease, wherein an increased expression level of the biomarker is indicative of an active phase.

17. The method of claim 16, wherein the biomarker is IL-6Rα and/or pSTAT3.

18. The method of claim 16 or 17, wherein the autoimmune or demyelinating disease is multiple sclerosis or relapsing-remitting multiple sclerosis.

19. An in vitro method for characterizing relapsing-remitting multiple sclerosis in a subject, comprising:
(a) obtaining a sample comprising CD4⁺ T cells from a first subject having relapsing-remitting multiple sclerosis;
(b) isolating CD4⁺ T cells from the sample to provide isolated CD4⁺ T cells;
(c) co-culturing the isolated CD4⁺ T cells with Treg cells obtained from a second subject not having relapsing-remitting multiple sclerosis;
(d) quantifying an amount of CD4⁺ T cell proliferation; and
(e) determining if CD4⁺ T cell proliferation is suppressed, wherein suppressed CD4⁺ T cell proliferation is indicative of an active phase of relapsing-remitting multiple sclerosis in the first subject.

20. A method for characterizing relapsing-remitting multiple sclerosis in a subject, comprising:
(a) obtaining a sample comprising CD4⁺ T cells from a subject having multiple sclerosis;
(b) exposing at least a portion of CD4⁺ T cells from the sample to IL-6 to provide stimulated CD4⁺ T cells;
(c) quantifying an amount of pSTAT3 in the stimulated CD4⁺ T cells; and
(d) determining if the amount of pSTAT3 is increased, wherein increased pSTAT3 is indicative of an active phase of relapsing-remitting multiple sclerosis in a subject.

21. Use of a compound effective to reduce phosphorylation of STAT3 in the manufacture of a medicament for use in the treatment of multiple sclerosis in a subject in need thereof.

22. A composition for treating multiple sclerosis comprising a composition selected from the group consisting of (a) an IL-6 inhibitor; (b) an IL-6Rα antagonist; (c) a sIL-6Rα antagonist; and/or (d) a STAT3 phosphorylation blocker, wherein the composition is administered for a time and in an amount effective to treat multiple sclerosis.
